(19) United States
(12) Patent Application Publication
(10) Pub. No.: US 2026/0020851 A1
(43) Pub. Date: Jan. 22, 2026

(54) SURGICAL DEVICE, METHOD OF MANUFACTURING, AND METHOD OF USING THE SURGICAL DEVICE

(71) Applicants: NICE SURGICAL SOLUTIONS PTE LTD, Singapore (SG); ALEXANDRA HEALTH PTE. LTD., Singapore (SG)

(72) Inventors: Fangzhou ZHOU, Singapore (SG); Zhi Qi YONG, Singapore (SG); Surendra Kumar MANTOO, Singapore (SG)

(21) Appl. No.: 19/340,273

(22) Filed: Sep. 25, 2025

(30) Foreign Application Priority Data
Mar. 27, 2023 (SG) 10202300825Q

(51) Int. Cl. A61B 17/04; A61B 17/00; A61B 17/06; A61B 17/072; A61B 17/3209; A61B 34/30

(52) U.S. Cl. CPC A61B 17/0469; A61B 17/0401; A61B 17/0491; A61B 17/06066; A61B 17/07207; A61B 17/3209; A61B 34/30; A61B 2017/00477; A61B 2017/00526; A61B 2017/00862; A61B 2017/00867; A61B 2017/0409; A61B 2017/0414; A61B 2017/0416; A61B 2017/0438; A61B 2017/0446; A61B 2017/0472; A61B 2017/0477; A61B 2017/06052; A61B 2017/061; A61B 2034/302

(57) ABSTRACT

Disclosed is a surgical device, the surgical device comprises a first jaw coupled with a second jaw to define a clamp for receiving a tissue; a purse-string suture profile formed along a first longitudinal portion of the clamp, the purse-string suture profile adapted to receive a pair of purse-string suture needles; a staple anvil formed on the first jaw and along a second longitudinal portion of the clamp; a stapling unit formed on the second jaw along the second longitudinal portion of the clamp; and an anchor attachment assembly operable to urge the pair of purse-string suture needles to deliver a suture through the tissue to form an anchorage.

### CROSS-REFERENCE TO RELATED APPLICATIONS

This application is a continuation of International Patent Application No. PCT/SG2024/050184, filed on Mar. 27, 2024, which claims priority to Singapore Application No. 10202300825Q, filed on Mar. 27, 2023, which are incorporated herein by reference in their entirety.

### TECHNICAL FIELD

The present disclosure relates to a surgical device suited for performing at least one of tissue resection, suture and/or stapling, a method of manufacturing, and a method of using the surgical device. In some aspects, the device may be a minimally invasive surgical instrument.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Tissue resection and stapling are usually achieved by using surgical devices, such as surgical staplers. Although the surgical staplers can perform cutting and stapling, they may not be used to form purse-string sutures, which is a necessary part of performing anastomosis.

In addition, current purse-string suture devices are unable to create automatic suturing to form a purse-string suture laparoscopically. Although current devices are designed to form purse-string sutures in an open surgery, the purse-string sutures may not be formed in a minimally invasive way. Further, current laparoscopic surgical devices are unable to perform purse-string sutures, particularly when laparoscopic instruments have restrained access to the surgical site through the cannula of a trocar.

Accordingly, there exists a need to provide a surgical device to address at least one of the aforementioned issues.

### SUMMARY

The disclosure was conceptualized to provide an improved surgical device for integrating tissue stapling, and/or resecting, and/or purse-string suture forming functions. Various embodiments of the disclosure relate to standalone surgical device and robotic surgical system instrument accessories for performing purse-string suture forming and/or stapling and/or resection of mammalian tissues, such as colons, intestines or rectum tissues. In various embodiments, the improved surgical device may be a laparoscopic device.

In some aspects, the surgical device enables a user, such as a medical practitioner, to perform tubular tissue resection and simultaneously staple seal the tubular tissue at one side and form a purse-string suture on the other side.

According to a first aspect of the disclosure, there is provided a surgical device comprising a first jaw coupled with a second jaw to define a clamp for receiving a tissue; a purse-string suture profile formed along a first longitudinal portion of the clamp, the purse-string suture profile adapted to receive a pair of purse-string suture needles; a staple anvil formed on the first jaw and along a second longitudinal portion of the clamp; a stapling unit formed on the second jaw along the second longitudinal portion of the clamp; and an anchor attachment assembly operable to urge the pair of purse-string suture needles to deliver a suture through the tissue to form an anchorage.

In various embodiments of the first aspect, the pair of purse-string suture needles each comprise a hollow interior, and the corresponding pair of anchors are each pre-attached to the suture, and wherein the pair of anchor pushers, the corresponding pair of anchors, and the suture are contained within the hollow interior to deliver suture bonded anchors through the tissue to form the anchorage.

In various embodiments of the first aspect, the surgical device further comprises a shaft, the shaft having a hollow interior for housing the pair of purse-string suture needles, the shaft pivotably coupled to the clamp in a manner such the clamp is rotatable with respect to the shaft. The shaft may be configured to hold a first part of the anchor attachment assembly.

In various embodiments of the first aspect, the surgical device further comprises a blade assembly, the blade assembly comprises an actuator and a blade, the blade slidably configured to move the clamp between an open position and a clamped position, and simultaneously extend along the longitudinal axis of the clamp, the blade operable to incise the tissue when the clamp is in the clamped position.

In various embodiments of the first aspect, the surgical device further comprises a suture fastener configured to facilitate closure of the purse-string suture loop from at least one end of a suture loop.

In various embodiments of the first aspect, the surgical device further comprises an articulation assembly positioned at the coupling of the shaft and the clamp, the articulation assembly comprising an articulation block positioned on either the first jaw and/or the second jaw. In some examples, the articulation block may be positioned on the second jaw, adjacent to the purse-string suture profile of the clamp, wherein the articulation block comprises articulation recesses leading into linear passages, the articulation recesses and the linear passages adapted to receive the pair of purse-string suture needles and guide the pair of purse-string suture needles to move towards and through the purse-string suture profile. In some embodiments, the purse-string suture needles may bend as a result of an articulation (of the clamp with respect to the shaft), and thereafter move in a linear manner towards and through the purse-string suture profile.

According to a second aspect of the disclosure, there is provided a robotic instrument comprising a connector interface for receiving the surgical device of the first aspect, wherein the surgical device is configured as an end effector.

According to a third aspect of the disclosure, there is provided a hand-held instrument comprising a connector interface for receiving the surgical device of the first aspect, wherein the surgical device is configured as an end effector.

According to fourth aspect of the disclosure, there is provided a stand-alone surgical device, integrally formed with a handle or a robotic instrument.

According to a fifth aspect of the disclosure, there is provided a method of manufacturing the surgical device of the first aspect, the method comprising the steps of providing a first jaw and a second jaw and coupling the first jaw and the second jaw to define a clamp; forming a purse-string suture profile along a first longitudinal portion of the clamp, the purse-string suture profile adapted to receive a pair of purse-string suture needles; forming a staple anvil on the first jaw and along a second longitudinal portion of the clamp; forming a stapling unit on the second jaw along the second longitudinal portion of the clamp; and providing an anchor attachment assembly, the anchor attachment assembly operable to urge the pair of purse-string suture needles to deliver a suture through a tissue to form an anchorage.

In various embodiments of the fifth aspect, the method further comprises, providing a shaft, the shaft having a hollow interior for housing the pair of suture needles, and pivotably coupling the shaft to the clamp in a manner such the clamp is rotatable with respect to the shaft.

In various embodiments of the fifth aspect, the method further comprises, providing a blade assembly, the blade assembly comprising an actuator and a blade, the blade slidably configured to move the clamp between an open position and a clamped position, and simultaneously extend along the longitudinal axis of the clamp, the blade operable to incise the tissue when the clamp is in the clamped position.

In various embodiments of the fifth aspect, the method further comprises, providing a suture fastener configured to facilitate closure of the purse-string suture loop from at least one end of a suture loop.

In various embodiments of the fifth aspect, the method further comprises, providing an articulation assembly configured to be positioned at the coupling of the shaft and the clamp, the articulation assembly comprising an articulation block positioned either the first jaw and/or the second jaw. The articulation block may be positioned adjacent to the purse-string suture profile of the clamp, and the articulation block comprises articulation recesses leading into linear passages, the articulation recesses and the linear passages adapted to receive the pair of purse-string suture needles and guide the pair of purse-string suture needles to bend as a result of an articulation (of the clamp with respect to the shaft) and thereafter move in a linear manner towards the purse-string suture profile.

According to a sixth aspect of the disclosure, there is provided a method of using the surgical device of the first aspect to perform suturing of a tubular tissue, comprising, positioning a tissue between the clamp; operating the blade to move the clamp to a clamped position; wherein in the clamped position, the tissue is compressed against the purse-string suture profile and the stapling unit; advancing the pair of purse-string suture needles (with suture, a pair of anchors, and a corresponding pair of anchor pushers) through the purse-string suture profile; suturing the tubular tissue with the purse-string suture needles; passing, using the pair of purse-string suture needles, through the anchor stopper of the anchor attachment assembly; depositing the pair of anchors with suture thereon through the anchor stopper; and retracting the pair of purse-string suture needles. In various embodiments, the purse-string suture needles may be retracted together with anchor pushers leaving suture engaged in the tissue and anchors engaged with anchor stopper adjacent to the tissue.

The dependent claims define some embodiments associated with the surgical device, the method of manufacturing the surgical device, and the method of using the surgical device to perform purse-string suturing, cutting and stapling of a tubular tissue.

### BRIEF DESCRIPTION OF THE DRAWINGS

The disclosure will be better understood with reference to the detailed description when considered in conjunction with the non-limiting examples and the accompanying drawings, in which:

FIG. 1 is a perspective view of a surgical device 100 or part thereof according to some embodiments of the disclosure.

FIGS. 2A and 2B show exposed sectional views of a shaft 101 of the surgical device 100 or part thereof according to some embodiments of the disclosure (blade is not shown).

FIGS. 3A and 3B show perspective views of the surgical device 100 with particular emphasis on the clamp 102 of the surgical device 100.

FIGS. 4A and 4B show perspective sectional view of the first longitudinal portion 106 of the clamp 102 comprising the purse-string suture profile 112, and FIG. 4C shows a sectional view of the purse-string suture profile 112.

FIGS. 5 to 10 show perspective side views of various embodiments of the pair of anchors 144.

FIGS. 11A to 12B show perspective views of various embodiments of the pair of purse-string suture needles 140.

FIGS. 13A to 16D show views of the first longitudinal portion 106 of the clamp 102, comprising various embodiments of the purse-string suture profile 112.

FIGS. 17 to 20 show perspective views of various embodiments of the anchor stopper 146.

FIGS. 21A and 21B show perspective sectional views of the second longitudinal portion 108 of the clamp 102 comprising the stapling unit 134 and the staple anvil 132.

FIG. 22 shows a front sectional view of the clamp 102 of the surgical device 100 according to various embodiments.

FIG. 23 shows a perspective sectional view of the clamp 102 of the surgical device 100 in a closed position, with staples loaded.

FIGS. 24A to 24D show perspective sectional views of the purse-string suture needles 140 being passed through the purse-string suture profile 112 and part of the anchor attachment assembly 142 according to some embodiments.

FIGS. 25A to 29H show sectional views of various embodiments of the decoupler assembly 150.

FIGS. 30A to 30C show sectional views of the surgical device 100, with particular emphasis on an articulation assembly 260 of the surgical device 100.

FIGS. 31A and 31B illustrate purse-string sutures formed using the surgical device.

FIG. 32 is a flowchart of a method 300 of manufacturing a surgical device according to some embodiments.

FIG. 33 is a flowchart of a method 400 using the surgical device to perform suturing of a tubular tissue according to some embodiments.

FIG. 34 illustrate various system diagrams of the surgical device as an attachment.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the disclosure. Other embodiments may be utilized and structural, and logical changes may be made without departing from the scope of the disclosure. The various embodiments are not necessarily mutually exclusive, as some embodiments can be combined with one or more other embodiments to form new embodiments.

The disclosure illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically described in exemplary embodiments and optional features, modification and variation of the disclosure embodied herein may be resorted to by those skilled in the art.

In the context of various embodiments, the articles “a”, “an” and “the” as used with regard to a feature or element include a reference to one or more of the features or elements. As used herein, the term “and/or” includes any and all combinations of one or more of the associated listed items.

While such terms as “first,” “second,” etc., may be used to describe various elements, such elements must not be limited to the above terms. The above terms are used only to distinguish one element from another, and do not define corresponding elements, for example, an order and/or significance of the elements. Without departing a scope of rights of the specification, a first element may be referred to as a second element, and similarly, the second element may be referred to as the first element.

Throughout the description, the term “tissue” as used herein, refers to various types of organ tissue, including but not limited to, a mammalian colon.

In the context of the present disclosure, the term “surgical device” includes part or portions thereof. For example, an end effector attachable to a handle or robotic arm may be regarded as a surgical device. The surgical device may further include minimally invasive surgery devices, such as, but not limited to, purse-string suture devices.

According to one aspect of the disclosure, there is provided a surgical device for performing tissue stapling, resection, and the formation of purse-string sutures. The surgical device is particularly suited, but not limited to anastomosis of tubular tissues. Such tubular tissue may include human colon tissues.

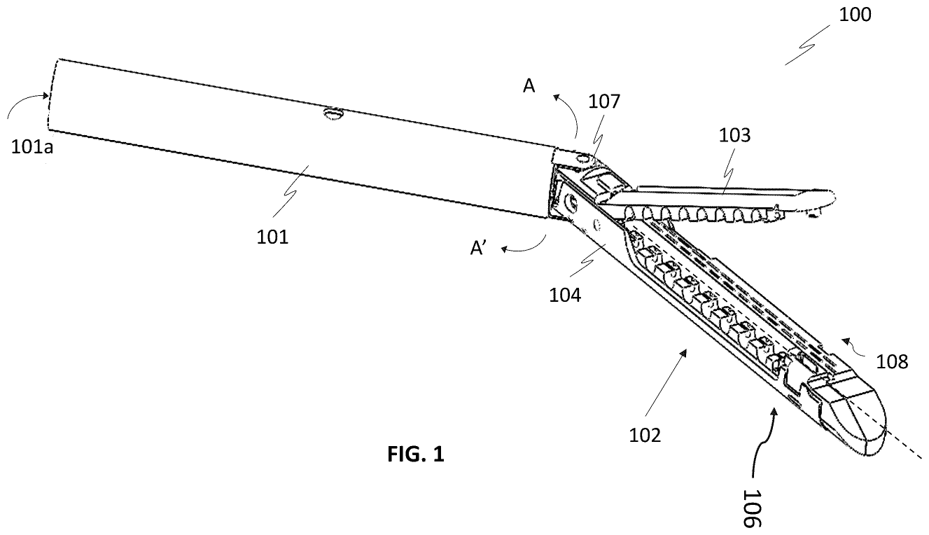

The various embodiments of which are discussed with reference to FIGS. 1 to 29G.

FIG. 1 shows a perspective view of an embodiment of the surgical device 100 comprising a clamp 102 and a shaft 101. The surgical device 100 may be implemented as an end effector in some embodiments involving a stand-alone device and/or an accessory to a robotic system and/or arm.

The clamp 102 of the surgical device 100 may comprise a first jaw 103 coupled with a second jaw 104 to define the clamp 102 for receiving a tissue (tissue not shown). The clamp 102 may comprise a first longitudinal portion 106 and second longitudinal portion 108.

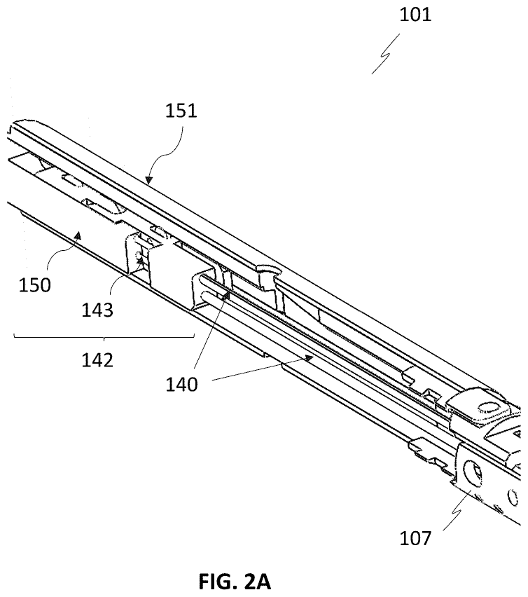

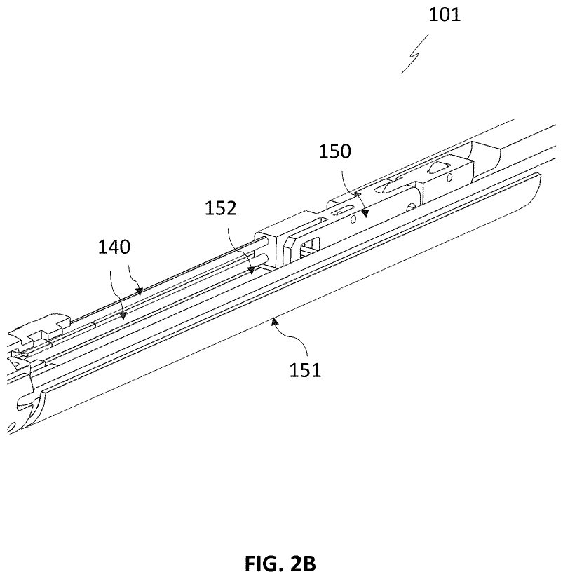

FIGS. 2A and 2B show exposed sectional views of the shaft 101. The shaft 101 may have a hollow interior for housing the pair of suture needles 140 and a portion of an anchor attachment assembly 142. The shaft 101 may be pivotably coupled to the clamp 102 in a manner such the clamp 102 is rotatable with respect to the shaft 101. The shaft 101 may be coupled to the clamp 102 via a hinge 107. The hinge 107 may be configured for the clamp 102 to rotate within a plane of the surgical device 100 as denoted by arrows labelled A-A′ in FIG. 1. In other words, the clamp 102 may rotate, e.g. articulate, with respect to the shaft 101 in the direction denoted by arrows labelled A-A′ in FIG. 1, which increases the dexterity of the surgical device 100.

The shaft 101 may include a shaft casing 151, which houses a part of the anchor attachment assembly 142 comprising a decoupler assembly 150 and a plurality of anchor pushers 143. The shaft 101 may also house the purse-string suture needles 140, an inner shaft 152, and a blade 158. In various embodiments, the decoupler assembly 150 may actuate the blade 158 and the purse-string suture needles 140.

The decoupler assembly 150 comprises various mechanisms to actuate the clamp 102 for performing the functions of tissue stapling, resection and/or purse-string suture formation. In some embodiments, the decoupler assembly 150 may be configured to move along a longitudinal axis of the shaft 101, and to urge an actuation mechanism or actuator toward the clamp 102 to move the first jaw 103 and the second jaw 104 towards each other.

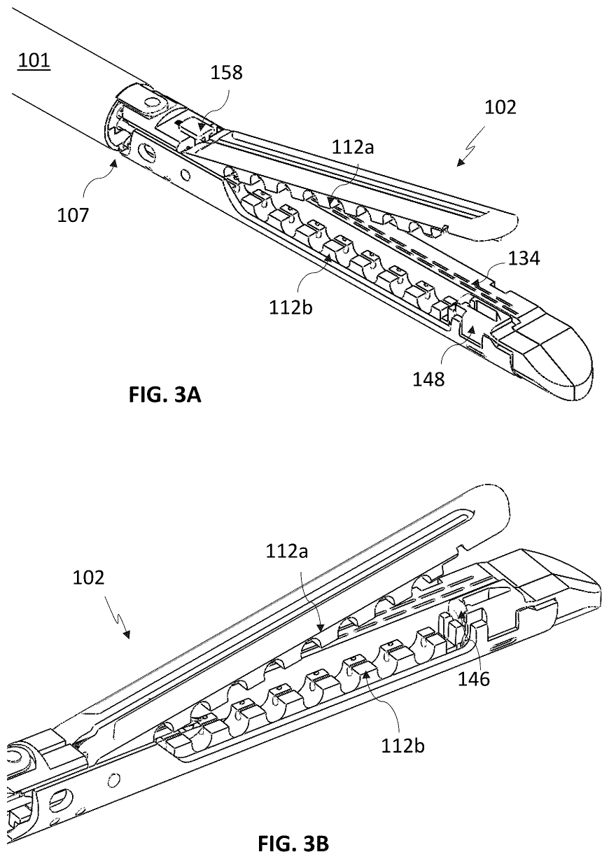

FIGS. 3A and 3B show perspective views of the surgical device 100 with particular emphasis on the clamp 102. The clamp 102 comprises the first jaw 103 and second jaw 104. The first jaw 103 and the second jaw 104 may move relative to each other. The purse-string suture profile 112 is formed on the first longitudinal portion 106, and the staple anvil 132 is formed on the second longitudinal portion 108.

A staple anvil 132 may be formed on the first jaw 103 and along the second longitudinal portion 108 of the clamp 102. A stapling unit 134 may be formed on the second jaw 104 along the second longitudinal portion 108 of the clamp 102, corresponding to the staple anvil 132 on the first jaw 103. An anchor attachment assembly 142 is operable to urge the pair of purse-string suture needles 140 to deliver a suture which may be coupled to a corresponding pair of anchors 144.

In some embodiments, the corresponding pair of anchors 144 may be coupled to the suture via or through an anchor stopper 146 to operably form a suture loop around the tissue.

In the embodiment shown in FIG. 2A, FIG. 2B, FIGS. 3A and 3B, the actuation mechanism may carry a blade 158 for incising the tissue. The decoupler assembly 150 may include a needle holder 190 coupled to the pair of purse-string suture needles 140, and an anchor pusher holder 196 coupled to a pair of anchor pushers 143. The decoupler assembly 150 may further carry the purse-string suture needles 140, the blade 158 and the pair of anchor pushers 143 to a distal end of the surgical device 100. The decoupler assembly 150 may be linked to the clamp 102 via an articulation joint (which may be in the form of the hinge 107). In some embodiments, the articulation joint (in the form of the hinge 107) may couple the shaft 101 and the clamp 102. In various embodiments, a part of the anchor attachment assembly, specifically, the decoupler assembly 150, may be configured to urge the purse-string needles 140, the blade 158 and the pair of anchor pushers 143 through the inner shaft 152 and clamp 102 via the articulation joint, the articulation joint forming a pivoting point between the clamp 102 and the shaft 101. In some embodiments, the decoupler assembly 150 may urge the purse-string suture needles 140, the blade 158 and the pair of anchor pushers 143, through the pivoting point, e.g. bend, between the shaft 101 and clamp 102. In some other embodiments, the clamp 102 may not be articulated with respect to the shaft 101, e.g. shaft 101 and clamp 102 are straight with respect to each other, and the decoupler assembly 150 may urge the purse-string suture needles 140, the blade 158 and the pair of anchor pushers 143, through the straight portion at which the shaft 101 is coupled to the clamp 102.

The anchor pushers 143 may be configured to keep the anchors (with suture attached to them) still relative to the purse-string suture needles 140 when the purse-string suture needles 140 are activated and advancing through the tissue during the suturing process.

In some embodiments, the anchor pushers 143 are adapted to urge the anchors 144, with suture attached to them, from the purse-string suture needles 140 when the decoupler assembly 150 moves to a predetermined position 111 after the purse-string suture needles 140 go through the anchor stopper 146.

At a first end, also referred to as proximal end 101a of the shaft 101, there may be a quick-connect connection interface for connection to an external device. The external device may be in the form of a handle or a robotic arm. The connection interface may allow the surgical device 100 to releasably connect to external device to facilitate usage and mechanical links can be established through the connection for the surgical device 100 to be operated in some embodiments, the mechanical links may include mechanical elements as known by a skilled person to provide mechanical linkage for the transmission of at least one of force, pressure, moment, torque, or load through the shaft 101 and clamp 102. In various embodiments, the force, pressure, moment, torque, or load may be transmitted through the decoupler assembly 150.

The first and second longitudinal portions 106, 108 may be connected to each other and be integrally molded or formed. In some embodiments, the first and second longitudinal portions 106, 108 may be formed separately and joined together using fasteners. In some embodiments, no fasteners may be required and the first and second longitudinal portions 106, 108 may be shaped and dimensioned to be force-fitted or shrink fitted with respect to each other. In some embodiments, there may be a longitudinal slot that allows the blade 158 to slide and keep the first jaw 103 and second jaw 104 at a predetermined controlled distance when the tissue is engaged. The second jaw 104 comprises the stapling unit 134 on the second longitudinal portion corresponding to the staple anvil 132 on first jaw 103.

The purse-string suture profile on the second jaw 104 is formed corresponding to the purse-string suture profile on the first jaw 103, such that, when the clamp 102 is in operation, that is, when clamping a tissue, the purse-string suture profile 112 is shaped and aligned to receive a pair of purse-string suture needles 140, which may in some embodiments, contain the pair of corresponding anchors 144 and the pair of anchor pushers 143.

At a distal end of purse-string profile shown in FIG. 3B, the anchor stopper 146 may be detachably held in alignment with a plurality of needle holes formed when the purse-string suture profile 112 in the first jaw 103 and the second jaw 104 are brought towards each other in a clamped position.

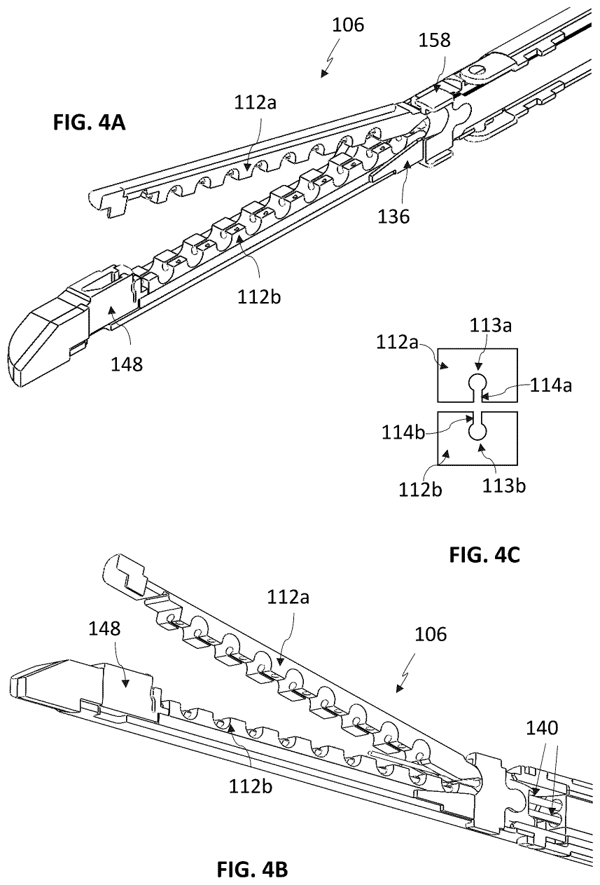

FIGS. 4A and 4B show perspective sectional views of the first longitudinal portion 106 of the clamp 102 comprising the purse-string suture profile 112, and the anchor chamber 148 located adjacent the purse-string suture profile 112. FIG. 4C shows a sectional view of the purse-string suture profile 112, having needles holes 113a, 113b adapted to receive the pair of purse-string suture needles 140, and a slit 114a, 114b for disengaging the suture from the purse-string suture profile 112.

Referring to FIGS. 4A and 4B, a purse-string suture profile 112 may be formed along the first longitudinal portion 106 of the clamp 102. The purse-string suture profile 112 may comprise an upper suture profile 112a formed along the first jaw 103, and a lower suture profile 112b formed along the second jaw 104, the upper suture profile 112a and lower suture profile 112b adapted to receive the purse-string suture needles 140 when the clamp 102 is closed by moving the first jaw 103 and/or the second jaw 104 towards each other. In various embodiments, the purse-string suture profile 112 on the second jaw 104 is formed corresponding to the purse-string suture profile on the first jaw 103, such that, when the clamp 102 is in operation, that is, when clamping a tissue, the purse-string suture profile 112 is shaped and aligned to receive the pair of purse-string suture needles 140 with the suture. In various embodiments, the purse-string suture profile 112 may comprise a stiff material, such as but not limited to plastic and/or metal. In some embodiments, the stiff material may include a biocompatible material.

The purse-string suture profile 112 on the first jaw 103 and the second jaw 104 may comprise a plurality of ridges and troughs such that when the first jaw 103 and second jaw 104 are brought together, the ridges and troughs define channels forming the needle holes. As shown in FIGS. 4A to 4C, the ridges of the upper suture profile 112a and the ridges of the lower suture profile 112b may each comprise a through hole 113a, 113b defining channels for forming the needle holes. The needles holes may be adapted to receive the purse-string suture needles 140. In some embodiments, the ridges of the upper suture profile 112a and the ridges of the lower suture profile 112b may further comprise a slit 114a, 114b, formed on the ridges, adjacent to the through hole 113a, 113b, i.e. needle holes, to allow the sutured tissue to disengage the purse-string suture profile 112 once the purse-string suture needles 140 are retracted. In some embodiments, the slits 114a, 114b may be formed such that the slit 114a of the upper suture profile 112a may be aligned to the slit 114b of the lower suture profile 112b. In some other embodiments, the slits 114a, 114b may be formed such that the slit 114a of the upper suture profile 112a may be offset, e.g. misaligned, with respect to the slit 114b of the lower suture profile 112b.

The plurality of needle holes, and the alignment thereof, facilitate the delivery of the pair of purse-string suture needles 140. The purse-string suture needles 140 will deliver the pair of anchors 144, which may include the suture pre-attached thereto, accurately through the anchor stopper 146. An anchor chamber 148 is located adjacent to the anchor stopper 146 in a distal position relatively further away from the hinge 107. The anchor chamber 148 provides a space for the pair of anchors 144 to be pushed out of the purse-string suture needles 140 and for the purse-string suture needles 140 to be deployed through the anchor stopper 146.

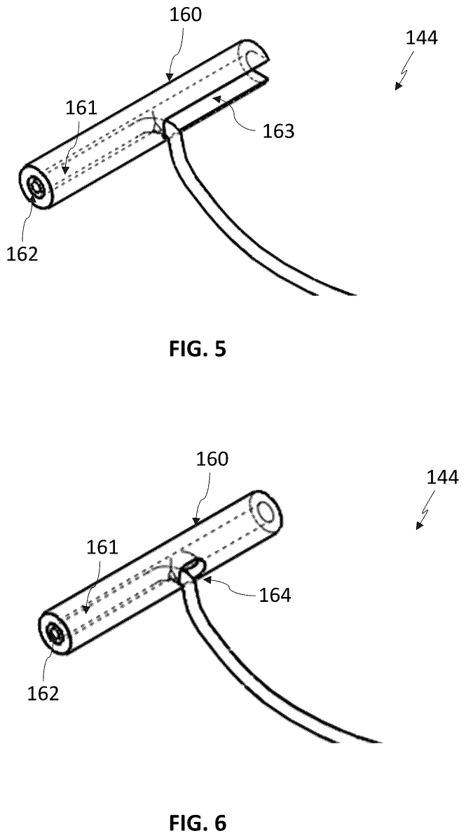

FIGS. 5 to 10 show perspective side views of various embodiments of the pair of anchors 144. The anchors 144 may include an anchor body 160 formed of a rigid material, such as but not limited to materials including plastics, and/or metals. In some embodiments, the rigid material may be elastic and be biocompatible.

In various embodiments, the suture may be pre-attached to the pair of anchors 144, and the purse-string suture needles 140 may therefore deliver the suture bonded anchors (contained in the hollow interior 121 of the purse-string suture needle 140), i.e. pair of anchors 144 having the suture threaded therethrough, through the tissue to achieve the suturing of the tissue, and the formation of the anchorage by depositing the pair of anchors 144 through the punctured, e.g. pierced, anchor stopper 146. The anchor body 160 includes at least one opening 162 arranged on an end of the anchor 144, and a hollow interior 161. The opening 162 and the hollow interior 161 may be operable to permit the suture to be threaded and bonded therethrough. It is contemplated that any apparatus suitable for being pre-attached to a suture and for forming the anchorage as described above, may be an anchor 144.

In various embodiments, the suture may be fixed to the anchor 144 to form suture bonded anchors, which may be facilitated by attachment means such as but not limited to gluing, welding and/or crimping. In some embodiments, the suture may be fixed to the anchor 144 by over-molding the suture with the anchor 144, or by molding the suture with the anchor 144. In other words, the suture and anchor may be integrally formed, e.g. as a “one-part” apparatus, or may be attached to each other, e.g. as a “two-part” apparatus. In various embodiments, the suture and the anchor 144 may include a same material, e.g. metal and/or plastic, or may each include different materials, e.g. the suture having a material that differs from the material of the anchor 144.

Referring to FIG. 5, the anchor body 160 may be tubular in shape, and may include a pre-made anchor slit 163 formed on a part of the anchor body 160. The anchor slit 163 may extend along a part of a length of the longitudinal axis of the anchor body 160, and may be shaped and dimensioned to receive the suture, and allow the suture to extend sideway from said anchor slit 163.

Referring to FIG. 6. the anchor body 160 may be tubular in shape, and may include a pre-made anchor opening 164 formed on a part of the anchor body 160. The anchor opening 164 may be formed in a middle part of the anchor body 160, and may be shaped and dimensioned to receive the suture, and allow the suture to extend from said anchor opening 164.

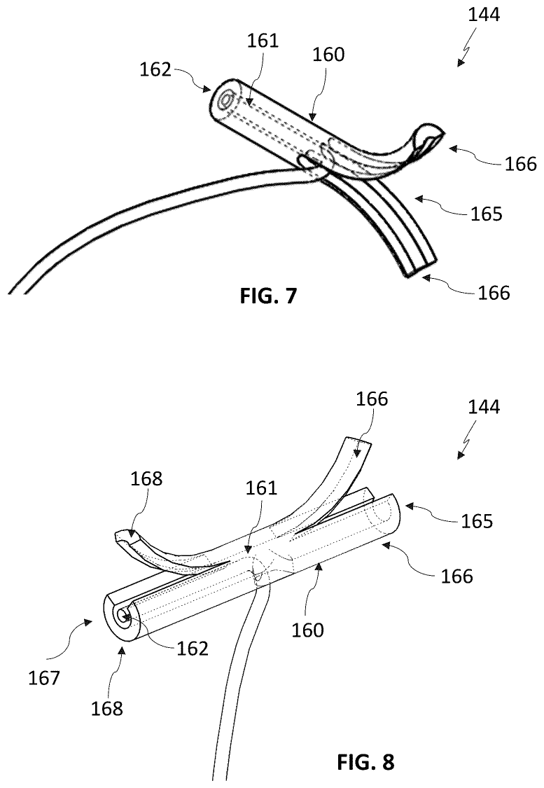

Referring to FIG. 7, the anchor body 160 may be tubular in shape, and may comprise a first anchor end 165 which is furcated. For example, a part of the anchor body 160 may be split such that first anchor end 165 forms a plurality of first anchor branches 166. In various embodiments, since the anchor body 160 is formed from a rigid and elastic material, the plurality of first anchor branches 166 may be compacted when the anchors 144 are contained within the hollow interior 121 of the purse-string suture needles 140, and may revert to its original form, after the anchors 144 disengage from the purse-string suture needles 140.

The anchor 144 shown in FIG. 8 may be based on the anchor 144 discussed with reference to FIG. 7, and may further include a second anchor end 167, positioned opposite the first anchor end 165. The second anchor end 167 may be furcated, and may form a plurality of second anchor branches 168. In various embodiments, the plurality of first anchor branches 166 and second anchor branches 168 may be compacted when the anchors 144 are contained within the hollow interior 121 of the purse-string suture needles 140, and may revert to its original form, after the anchors 144 disengage from the purse-string suture needles 140.

While two first anchor branches 166, and two second anchor branches 168 have been depicted in FIGS. 7 and 8, embodiments of the disclosure are not limited thereto, and the first anchor end 165 and the second anchor end 167 may each include more than two first anchor branches 166 and second anchor branches 168, respectively.

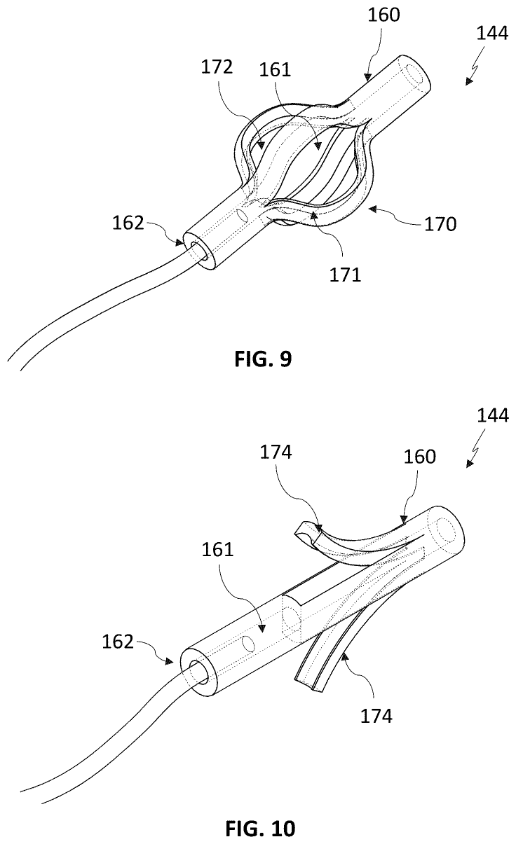

Referring to FIG. 9, the anchor 144 may include a pre-made anchor protrusion 170 extending from a part of the anchor body 160. The anchor protrusion 170 may extend from a middle part of the anchor body 160, and may include a plurality of anchor grids 171 which bulge outwards from the anchor body 160 and encase the hollow interior 161 of the anchor body 160. In various embodiments, the suture may be threaded through the opening 162 formed on an end of the anchor body 160, and may exit from another opening formed on the opposing end. It is contemplated that the suture may be exit from a portion 172 of the anchor protrusion 170, for example, between respective anchor grids 171 of the anchor protrusion 170. In various embodiments, since the anchor body 160 is formed from a rigid and elastic material, the anchor protrusion 170 may be compacted when the anchors 144 are contained within the hollow interior 121 of the purse-string suture needles 140, and may revert to its original form, after the anchors 144 disengage from the purse-string suture needles 140.

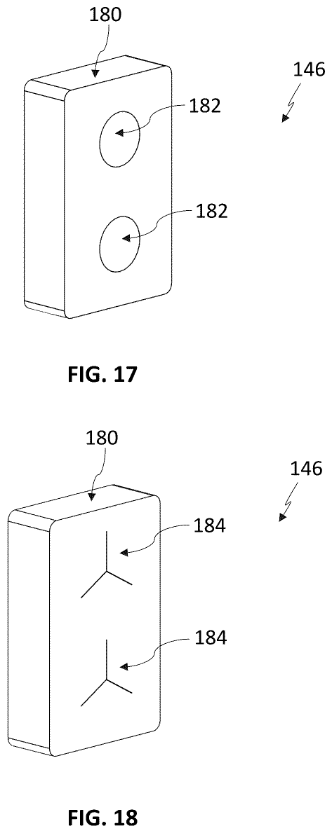

Referring to FIG. 10, the anchor 144 may include a plurality of pre-made anchor legs 174 extending from a part, for example, a middle part of the anchor body 160. The plurality of anchor legs 174 may extend away from the anchor body 160 and may form hooks which may aid in the formation of the purse-string suture loop. In various embodiments, the plurality of anchor legs 174 may be compacted when the anchors 144 are contained within the hollow interior 121 of the purse-string suture needles 140, and may revert to its original form, after the anchors 144 disengage from the purse-string suture needles 140.

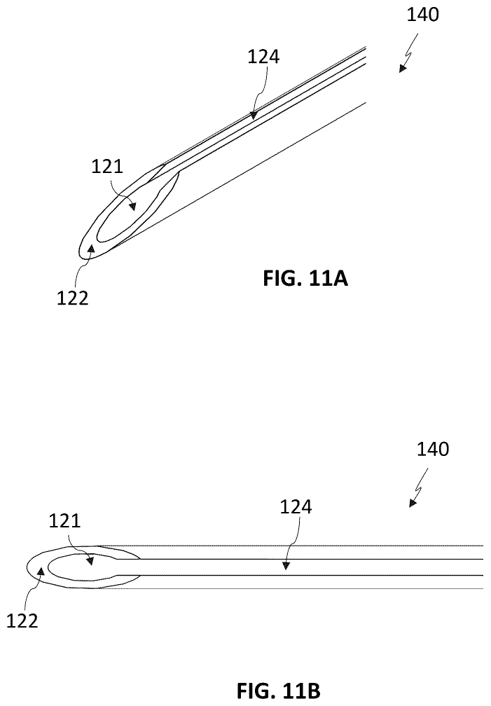

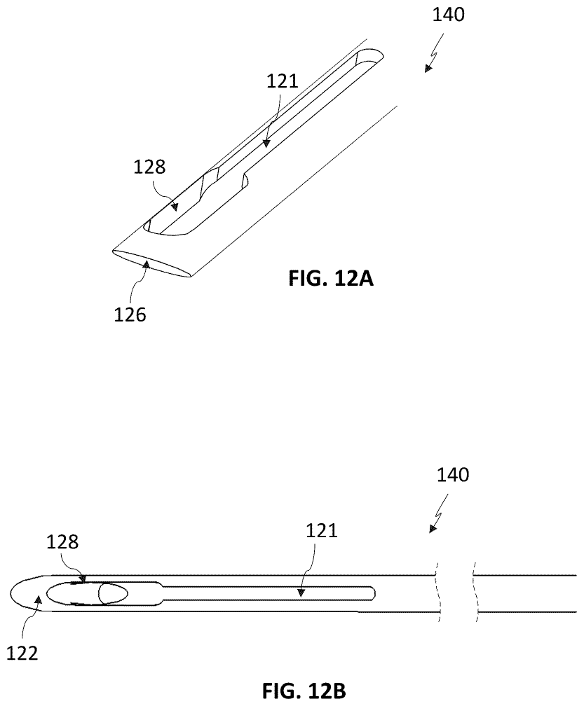

FIGS. 11A to 12B show perspective views of various embodiments of the pair of purse-string suture needles 140. FIG. 11A shows a perspective side view and FIG. 11B shows a perspective top view, of an embodiment of the purse-string suture needle 140. In various embodiments, the purse-string suture needles 140 may be comprise a hollow interior 121, which may contain the pair of anchor pushers 143, and the pair of anchors 144 discussed with reference to FIGS. 5 to 10. In some embodiments, the pair of anchors 144 may include the suture pre-attached to said anchors 144, which may also be referred to as suture bonded anchors in the disclosure. In an embodiment, the purse-string suture needles 140 may comprise cannular needles.

As shown in FIGS. 11A and 11B, the purse-string suture needles 140 may include a pointed tip 122 which is sharp to pierce the tissue and the anchor stopper 146, e.g. any one of the pre-made holes 182, the Y-shaped slits 184, the first section 186, of the anchor attachment assembly 142. The purse-string suture needle 140 may further include a slit 124 extending along at least, a part of a longitudinal axis, e.g. length, of the purse-string suture needle 140, and may be connected to the pointed tip 122 of the purse-string suture needle 140. In various embodiments, the slit 124 may be formed on the side opposite to the pointed tip 122 of the purse-string suture needle 140. In some embodiments, the slit 124 may extend along a full length of the purse-string suture needle 140. In some other embodiments, the slit 124 may extend along a half, or less than half of the length of the purse-string suture needle 140. Alternatively, the purse-string suture needles 140 may not include the slit 124 extending on the length of the purse-string suture needles 140.

FIG. 12A shows a perspective side view and FIG. 12B shows a perspective top view, of another embodiment of the purse-string suture needle 140. In some embodiments, the purse-string suture needles 140 may include a slanted tip 126, which is sharp to pierce the tissue and the anchor stopper 146 of the anchor attachment assembly 142. The slanted tip 126 may facilitate the delivery of the suture bonded anchors through the tissue and the anchor stopper 146 by allowing the suture bonded anchors to freely slide and disengage from the purse-string suture needles 140 in a transverse manner. In some embodiments, a slot 128 may be formed proximal to the slanted tip 126, to permit the release of the suture bonded anchors from the purse-string suture needles 140.

In various embodiments, the pair of purse-string suture needles 140 and/or the pair of anchor pushers 143 may be formed on, formed of or from a super-elastic material, such as but not limited to nickel-titanium alloy (nitinol). The super-elasticity of the purse-string suture needles 140 and/or the pair of anchor pushers 143 allows the purse-string suture needles 140 and/or the pair of anchor pushers 143 to restore to their original shape once after they are urged through (e.g. in some embodiments, bent through) the articulation joint formed between the shaft 101 and the clamp 102, and allows the purse-string suture needles 140 moving along a linear, e.g. straight, manner as it passes through the purse-string suture profile 112 of the clamp 102. The use of the super-elastic material also prevents the purse-string suture needles 140 and/or the pair of anchor pushers 143 from exceeding their respective elastic limit and preventing them from permanent deformation. In some embodiments, the anchor pushers 143 may not include a super-elastic material. Due to the smaller cross-sectional area of the anchor pushers 143 (relative to the purse-string suture needles 140), the purse-string suture needles 140 may continue to move in a substantially linear manner as it passes through the purse-string suture profile 112 of the clamp 102, even if the anchor pushers 143 are strained beyond their elastic limit.

It is appreciable that the plurality of ridges and troughs of the purse-string suture profile 112 facilitate the clamping of the tissue, and when the first jaw 103 and the second jaw 104 are moved towards each other to clamp the tissue, the defined clamp compresses and guides the tissue such that the tissue deforms and conforms to the purse-string suture profile 112. For example, the ridges clamp and compress the tissue, and the tissue is displaced by the ridges, and is accommodated in the spatial cavities of the trough. This facilitates the purse-string suture needles 140 to penetrate the tissue through the troughs and bypass the tissue at the ridges.

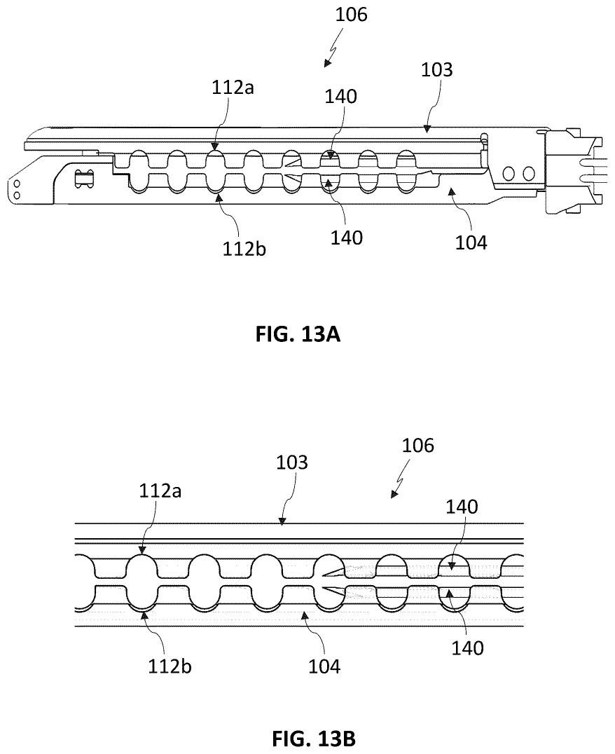

FIGS. 13A to 16D show views of the first longitudinal portion 106 of the clamp 102, comprising various embodiments of the purse-string suture profile 112.

Referring to FIGS. 13A and 13B which show side views of an exemplary embodiment of the purse-string suture profile 112, each ridge of the plurality of ridges of the upper suture profile 112a may be formed in alignment with a corresponding ridge of the plurality of ridges of the lower suture profile 112b, and each trough of the plurality of troughs of the upper suture profile 112a may be formed in alignment with a corresponding trough of the plurality of troughs of the lower suture profile 112b. That is, the ridges and troughs of the upper suture profile 112a and the lower suture profile 112b may be aligned and may be symmetrical to each other.

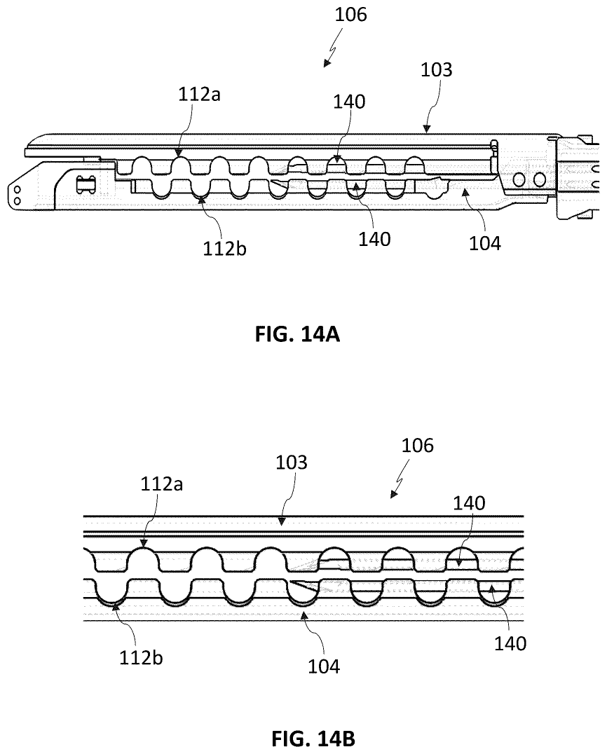

In various embodiments, each ridge of the plurality of ridges and each trough of the plurality of troughs of the upper suture profile 112a, may be formed to be misaligned and offset with respect to a corresponding ridge of the plurality of ridges and a corresponding trough of the plurality of troughs, respectively, of the lower purse-string suture profile 112b. In the exemplary embodiment as shown in FIGS. 14A and 14B, which show the side views of the purse-string suture profile 112, each ridge of the plurality of ridges of the upper suture profile 112a, may be formed in alignment with a corresponding trough of the plurality of troughs of the lower suture profile 112b, and each trough of the plurality of troughs of the upper suture profile 112a, may be formed in alignment with a corresponding ridge of the plurality of ridges of the lower suture profile 112b. In other words, the plurality of ridges of the upper suture profile 112a may be aligned with the plurality of troughs of the lower suture profile 112b.

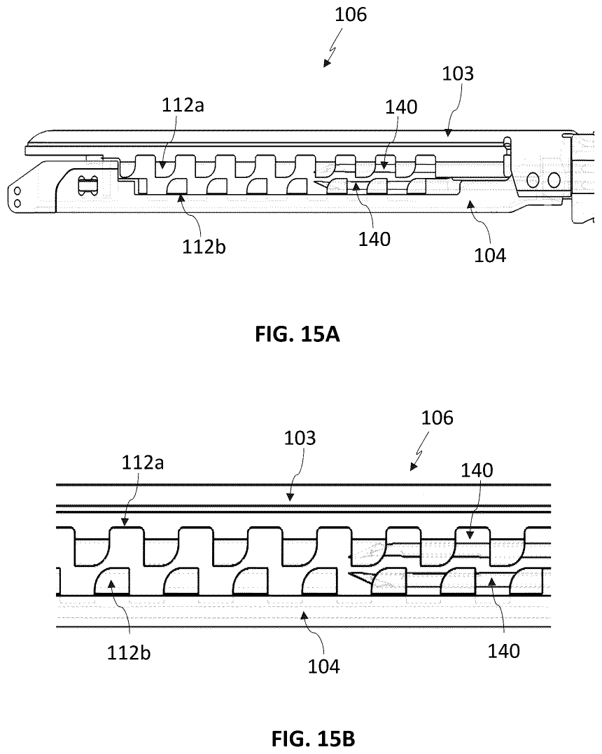

Referring to FIGS. 15A and 15B, which show side views of an exemplary embodiment of the purse-string suture profile 112, each ridge of the plurality of ridges of the upper suture profile 112a and the lower suture profile 112b may comprise a ridge surface having a substantially quadrant-shaped profile, e.g. a wedged ridge surface. The quadrant-shaped profile of the ridge surface may pinch the tissue and at the same time, the ridge will deform slightly due to the side load resultant from the quadrant-shaped profile, and accordingly, the quadrant-shaped surface will provide further compression of the tissue, as it deforms into the cavities of the troughs. In some embodiments, the lower suture profile 112b may comprise any shape suitable for facilitating the pinching and compression of tissue, in operation.

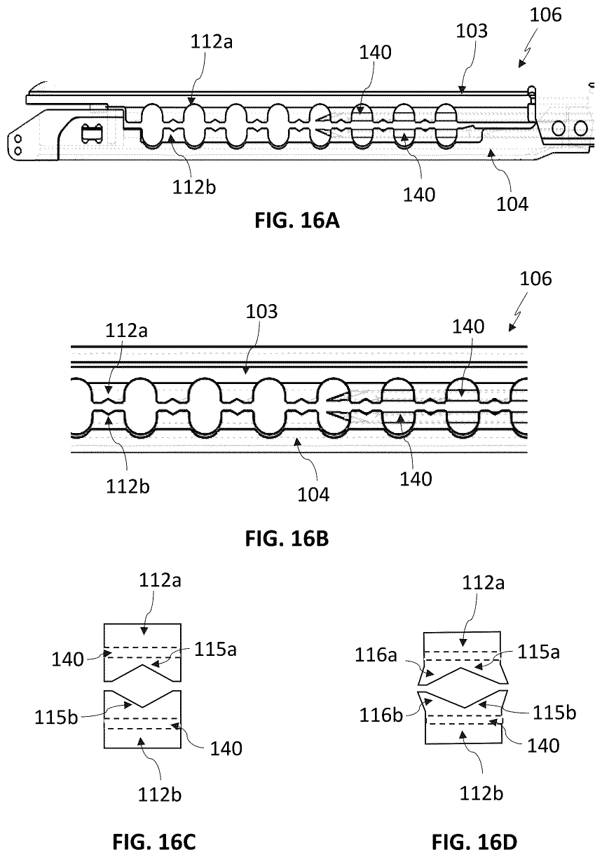

FIGS. 16A and 16B show side views of an exemplary embodiment of the purse-string suture profile 112, and FIGS. 16C and 16D show side views of the ridge surface of the purse-string suture profile 112 of FIGS. 16A and 16B, as the ridge surface clamps onto the tissue. Referring to FIGS. 16A to 16C, each ridge of the plurality of ridges of the upper suture profile 112a and the lower suture profile 112b may comprise a ridge surface having a V-shaped depression 115a, 115b formed on a part of the ridge surface. Referring to FIG. 16D, as the tissue is clamped between the V-shaped depression 115a, 115b, the sides of the ridge surface adjacent to the V-shaped depression 115a, 115b may deflect and extend towards a corresponding adjacent trough of the plurality of troughs, thereby pushing the tissue into the cavities of the troughs. In some embodiments, the V-shaped depression 115a, 115b may be substantially V-shaped and may include similar shapes facilitating the same function, e.g. a U-shape.

In various embodiments, the blade assembly may comprise the blade 158 and an actuator for actuating the blade 158. The blade 158 may be positioned between the first longitudinal portion 106 and the second longitudinal portion 108, and configured to slidably move along the longitudinal axis of the clamp 102 by the movement of the anchor attachment assembly 142, specifically, the decoupler assembly 150. When the blade 158 is pushed distally away from the hinge 107, and the movement of the blade 158 will close either of the first or second jaws 103, 104 to the other and cut the tissue clamped between the first jaw 103 and the second jaw 104. The actuator may comprise a plurality of metal straps, each metal strap attached to an adjacent metal strap, and each configured to slide relative to each other to reduce the load or force required to articulate the blade 158.

In various embodiments, the anchor stopper 146 may be positioned on either the first jaw 103 or the second jaw 104. It is further appreciable or contemplated that the anchor chamber 148 can also be positioned on either the first jaw 103 or the second jaw 104. In some embodiments, the anchor chamber 148 may be located on a different jaw relative to the anchor stopper 146.

FIGS. 17 to 20 show perspective side views of various embodiments of the anchor stopper 146. The anchor stopper 146 may include an anchor stopper body 180 which comprise a portion thereof that is shaped and dimensioned to receive the purse-string suture needles 140, such that the purse-string suture needles 140 can pass through the anchor stopper 146 without puncturing the anchor stopper material.

Referring to FIG. 17, the anchor stopper body 180 may include a pair of pre-made holes 182, shaped and dimensioned to receive the purse-string suture needles 140. The suture bonded anchors may therefore be deposited within the anchor chamber 148 after the purse-string suture needles 140 pass through the anchor stopper 146, and may interact with the pre-made holes 182 and anchor stopper body 180 such that the suture bonded anchors will not be able to retreat from the pre-made holes 182, e.g. exit the anchor chamber 148, once they have been deployed and accordingly, form the purse-string suture loop.

Referring to FIG. 18, the anchor stopper body 180 may include a pair of Y-shaped slits 184, shaped and dimensioned to receive and be pierced by the purse-string suture needles 140. The suture bonded anchors may interact with the Y-shaped slits 184 and anchor stopper body 180 to form the purse-string suture loop. In various embodiments, as the purse-string suture needles 140 are retracted from the anchor chamber 148, the Y-shaped slits 184 may close to prevent the deposited anchors 144 from being retracted along with the purse-string suture needles 140, once the anchors 144 have been deployed. In other words, the Y-shaped slits 184 may behave similar to a one-way valve to retain the deployed anchors 144 in the anchor chamber 148.

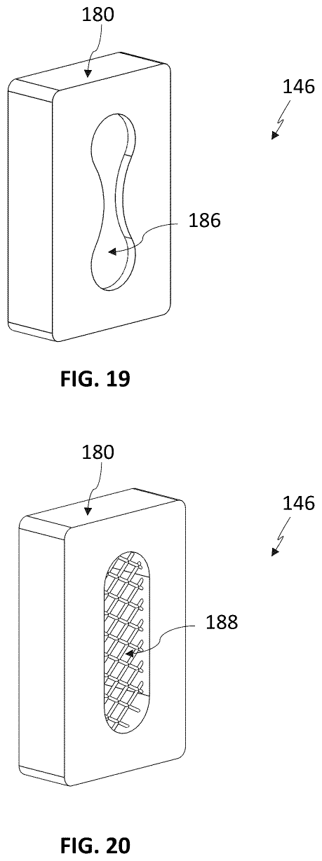

Referring to FIG. 19, the anchor stopper body 180 may include a first section 186 formed thereon, which is shaped and dimensioned to receive and be pierced by the purse-string suture needles 140. The first section 186 may be formed of a first anchor stopper material, which may differ from the anchor stopper material of the anchor stopper body 180. The first anchor stopper material may have a penetration resistance that is lower than the anchor stopper material of the anchor stopper body 180. In other words, the purse-string suture needles 140 may easily pierce and penetrate through the first section 186. In various embodiments, the first anchor stopper material may include shallow scribes formed on a rigid or flexible material, and accordingly, said material may be structurally weakened by the scribes. In various embodiments, the first section 186 may include the same material as the anchor stopper body 180, and the structure of the first section 186 is formed to be more penetrable than the anchor stopper body 180, by means of having a reduced thickness of the material, scribes, slit cut, and/or other means for weakening the material of the first section 186. In some embodiments, the material of the anchor stopper body 180, e.g. second anchor stopper material, may be adapted to receive and be pierced by the purse-string suture needles 140.

The anchor stopper 146 shown in FIG. 20 may be based on the anchor stopper 146 discussed with reference to FIG. 19. As shown in FIG. 20, the first section 186 having the first anchor stopper material may include a mesh structure having a penetration resistance lower than the anchor stopper material of the anchor stopper body 180. Each mesh grit of the mesh structure may be configured to reduce in size, as the purse-string suture needles 140 are retracted from the anchor chamber 148, thereby preventing the deposited anchors 144 from being retracted along with the purse-string suture needles 140, once the anchors 144 have been deployed.

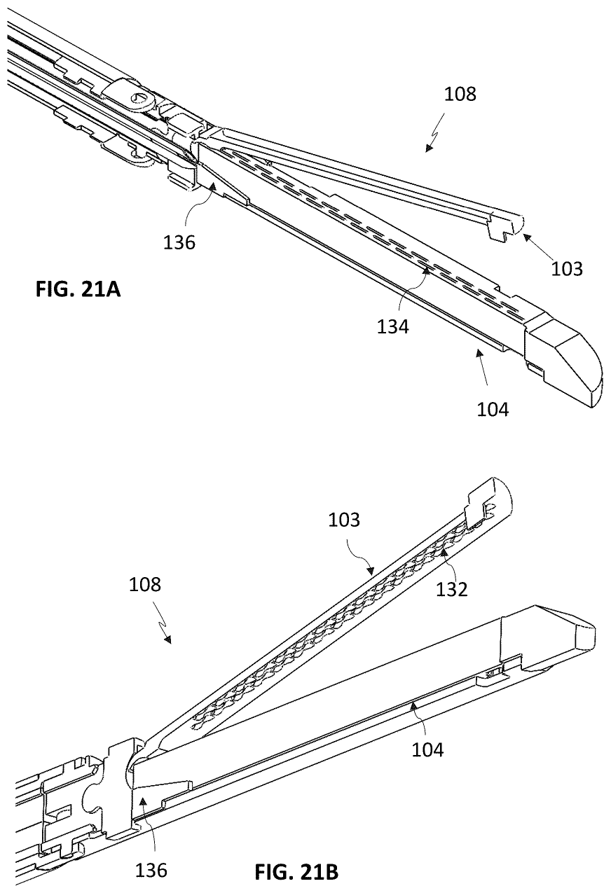

FIGS. 21A and 21B show perspective sectional views of the second longitudinal portion 108 of the clamp 102 comprising the stapling unit 134 and the staple anvil 132.

The stapling unit 134 may consist of at least one staple cartridge within which at least one row of staples is placed parallel to the longitudinal axis of the surgical device 100. In some embodiments, the stapling unit 134 may be similar to the linear stapler cartridges in the market.

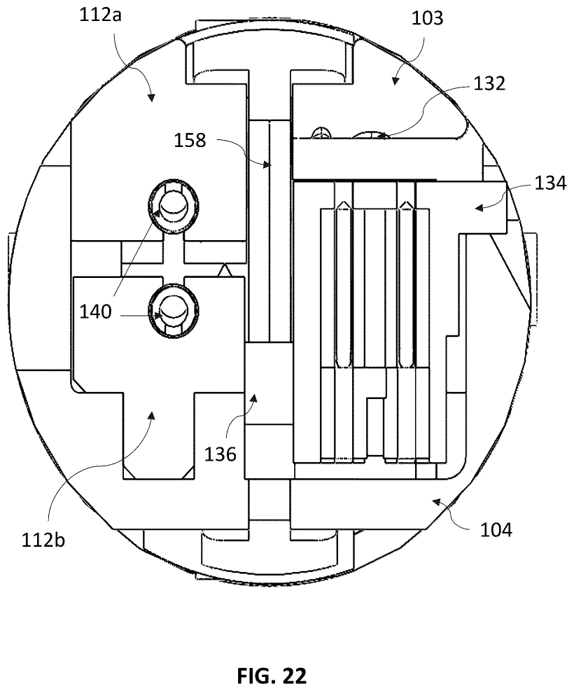

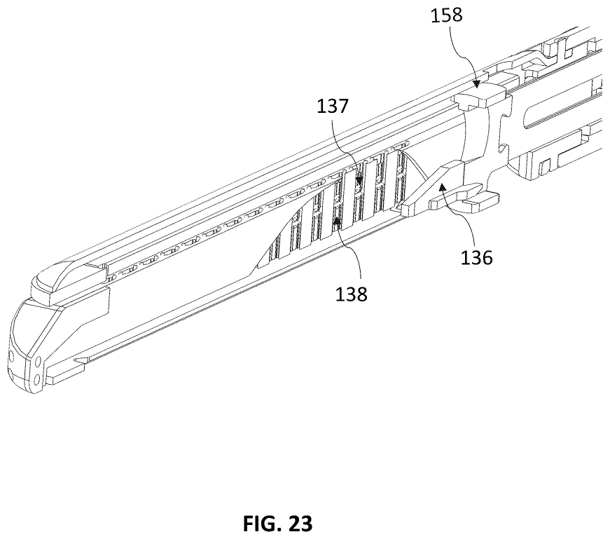

FIG. 22 shows a front sectional view of the clamp 102 of surgical device 100 according to various embodiments. FIG. 23 shows a perspective sectional view of the clamp 102 of the surgical device 100 in a closed position, with staples loaded. A staple ramp 136 may be positioned between the first longitudinal portion 106 and the second longitudinal portion 108 and may be positioned in front of the blade 158. Upon actuation of the blade 158, the blade 158 may drive the staple ramp 136 to slide along the longitudinal axis of the clamp 102 to discharge a staple 137, via a staple pusher 138, out of a cartridge of the stapling unit 134. The discharged staple will then be plied against the staple anvil 132 and deformed when the staple 137 penetrates the tissue to staple two layers of tissue together. As shown in FIG. 23, the staple ramp 136 may include a wedged surface positioned below the stapler pusher 138 and staple 137 of the stapling unit 134, the wedged surface operable to push the stapler pusher 138 and discharge the staple 137.

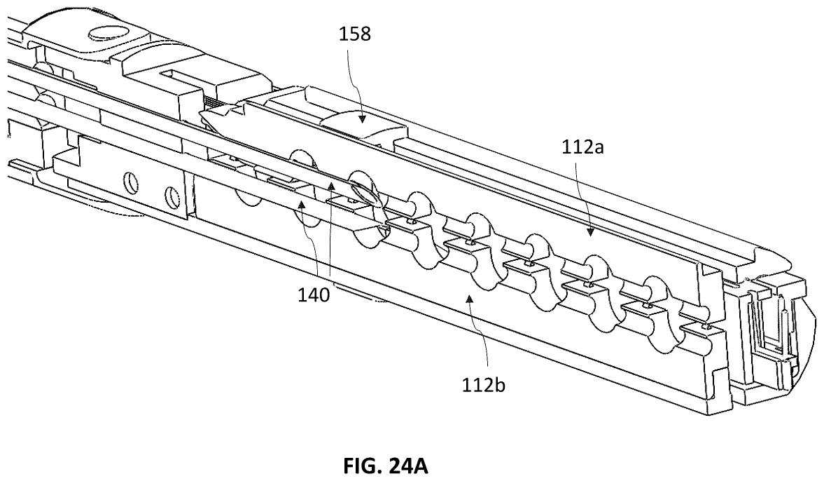

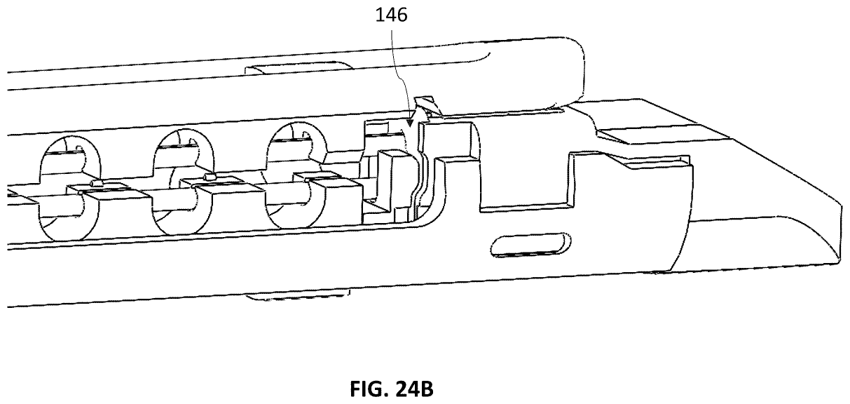

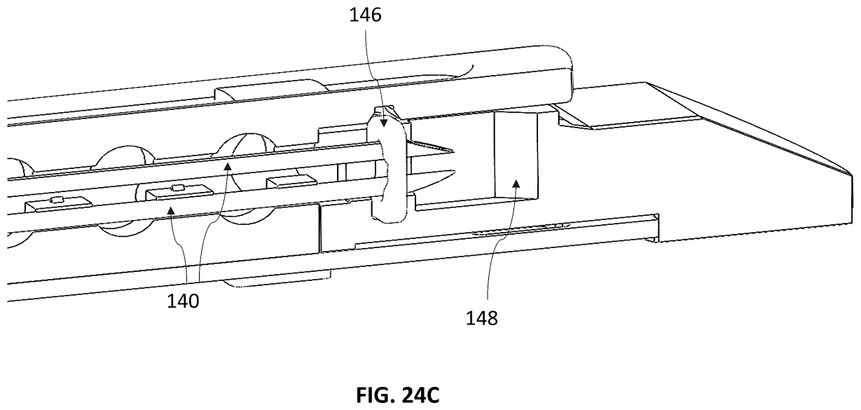

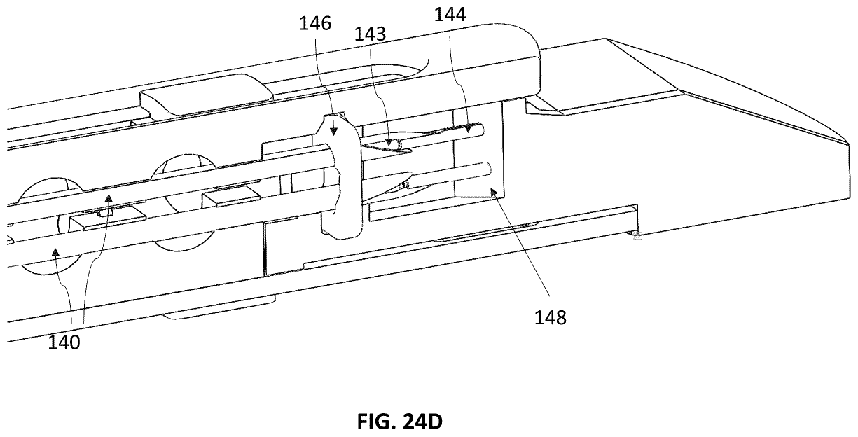

FIGS. 24A to 24D show perspective sectional views of the purse-string suture needles 140 being passed through the purse-string suture profile 112 and part of the anchor attachment assembly 142 according to some embodiments.

FIG. 24A shows the clamp 102 of surgical device 100 in a closed position with the purse-string suture needles 140 approaching the distal end of the surgical device 100. In various embodiments, the decoupler assembly 150 is operable to carry the pair of purse-string suture needles 140, containing the pair anchor pushers 143, the corresponding pair of anchors 144 having the suture pre-attached thereto, to the distal end of the surgical device 100, for formation of the purse-string suture loop.

FIG. 24B shows the pair of purse-string suture needles 140 passing through the anchor stopper 146. FIG. 24C shows a sectional view of FIG. 24B.

FIG. 24D shows the anchor pusher 143 operable to push the anchors 144 out of the purse-string suture needles 140, and into the anchor chamber 148.

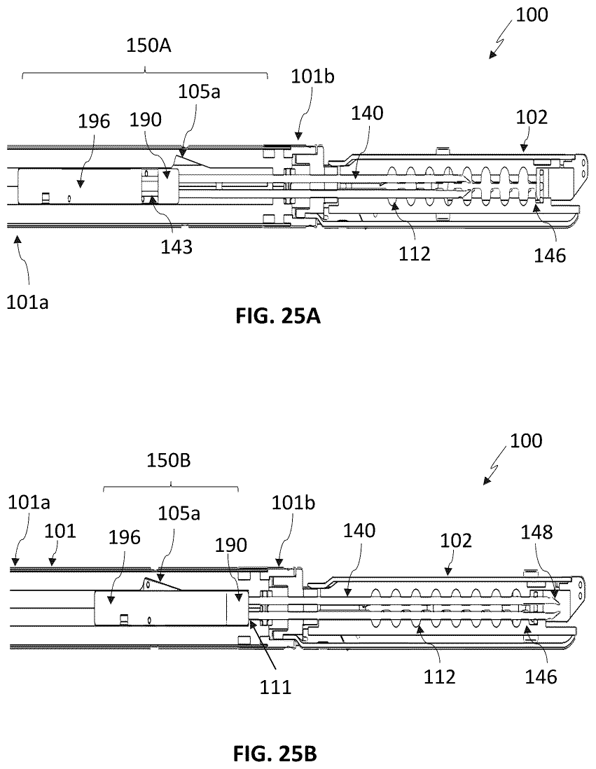

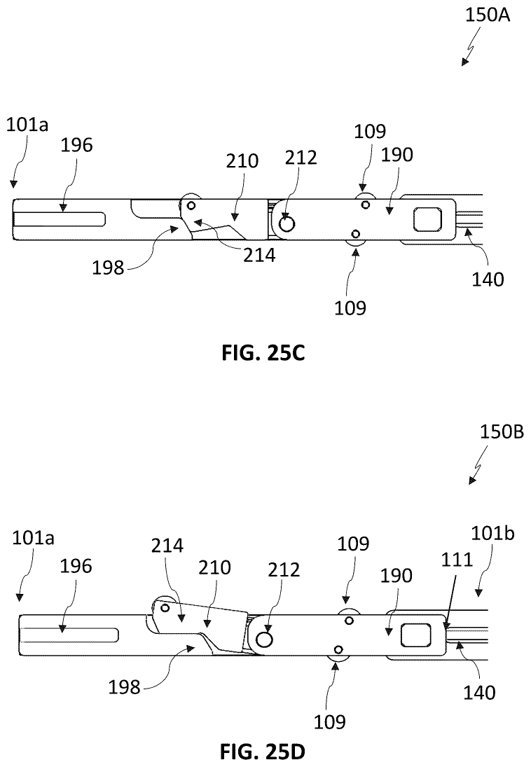

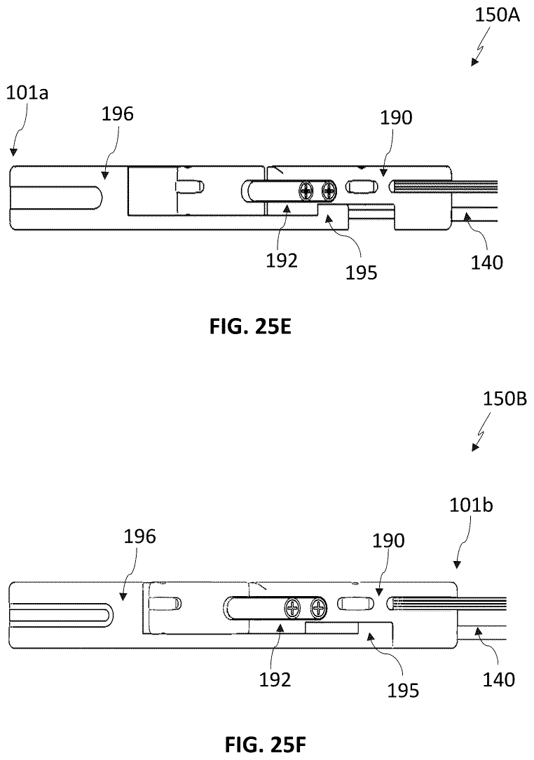

FIGS. 25A to 29H show sectional views of various embodiments of the decoupler assembly 150. The decoupler assembly 150 includes a needle holder 190 coupled to the pair of purse-string suture needles 140, and an anchor pusher holder 196 coupled to the pair of anchor pushers 143. In various embodiments, the needle holder 190 may be fixed to the pair of purse-string suture needles 140, and the anchor pusher holder 196 may be fixed to the pair of anchor pushers 143, using appropriate means for coupling the needle holder 190 to the pair of purse-string suture needles 140, and the anchor pusher holder 196 to the pair of anchor pushers 143. Non-limiting examples of such means include welding, mechanical fitting, over-molding of the needle holder 190 to the pair of purse-string suture needles 140 and/or the anchor pusher holder 196 to the pair of anchor pushers 143, and adhesive means.

In the context of the present disclosure, the term “proximal to” as used herein, may refer to the needle holder 190 being positioned directly adjacent to anchor pusher holder 196. The term “proximal to” may also refer to the needle holder 190 being positioned in close proximity, to the anchor pusher holder 196, and having one or more elements positioned in between the needle holder 190 and the anchor pusher holder 196.

FIGS. 25A and 25B show side sectional views of the surgical device 100 with particular emphasis on the decoupler assembly 150, in the coupled position 150A (FIG. 25A), and the decoupled position 150B (FIG. 25B), in accordance with an embodiment of the disclosure. FIGS. 25C and 25D show side views of the decoupler assembly 150 in the coupled position 150A (FIG. 25C) and the decoupled position 150B (FIG. 25D), from a side of decoupler assembly 150. FIGS. 25E and 25F show other side views of the decoupler assembly 150 in the coupled position 150A (FIG. 25E) and the decoupled position 150B (FIG. 25F), from another side of decoupler assembly 150. FIGS. 25C and 25E corresponds to the views of the decoupler assembly 150 as shown in FIG. 25A, and FIGS. 25D and 25F corresponds to the view of the decoupler assembly 150 as shown in FIG. 25B, respectively.

Referring to FIG. 25A to 25D, the decoupler assembly 150 may include a first spacer 210 pivotably coupled to the needle holder 190 via a first hinge 212, which is positioned adjacent to the needle holder 190. The anchor pusher holder 196 may be positioned directly adjacent to the first spacer 210 in the coupled position 150A. In other words, the needle holder 190 and the anchor pusher holder 196 may be spaced from each other via the first spacer 210. This facilitates the loading of the pair of anchor pushers 143, and the corresponding pair of anchors 144 (with the suture pre-attached thereto), in the hollow interior 121 of the purse-string suture needles 140.

As shown in FIGS. 25C and 25D, the anchor pusher holder 196 may include a wedged-shaped portion 198, and the first spacer 210 may include a first spacer portion 214 shaped and dimensioned to interact with the wedged-shaped portion 198 of the anchor pusher holder 196. In some embodiments, the inner shaft 152 of shaft 101 may include a first recess 105a formed along the longitudinal axis, e.g. length, of the shaft 101. The first recess 105a may be a void or a cut-out formed in the inner shaft 152, and may be shaped and dimensioned to receive at least, a part of the first spacer 210.

In operation, for example, upon application of an axial load or force to cause the pair of purse-string suture needles 140 to slide to a distal end of the surgical device 100, the decoupler assembly 150 may be operable to slide to the predetermined position 111 along the longitudinal axis of the shaft 101, and the decoupler assembly 150 may slide the purse-string suture needles 140 from the proximal end 101a of the inner shaft 152, to the distal end 101b of the inner shaft 152. The predetermined position 111 may therefore refer to the distal end 101b, e.g. end portion, of the inner shaft 152 of shaft 101, and at which the purse-string suture needles 140 pass through the anchor stopper 146. In various embodiments, the predetermined position 111 may refer to an end of the tunnel defined by an interior of the inner shaft 152.

In various embodiments, the wedged-shaped portion 198 of the anchor pusher holder 196 may push the first spacer portion 214, to urge the needle holder 190 to the predetermined position 111. In some embodiments, the restriction of the tunnel of the inner shaft 152 of the shaft 101 may prevent the first spacer 210 from rotating about the first hinge 212 as it moves towards the predetermined position 111. In some embodiments, a rolling element 109 may be formed on the needle holder 190 and/or the anchor pusher holder 196 to reduce friction as the needle holder 190 and/or the anchor pusher holder 196 moves along the longitudinal axis of the shaft 101.

As shown in FIGS. 25C and 25D, at the predetermined position 111, the decoupler assembly 150 may move from the coupled position 150A into the decoupled position 150B. Due to the first recess 105a, the first spacer 210 is operable to pivot, e.g. rotate about the first hinge 212, into the first recess 105a. Since at least a part of the first spacer 210 is no longer between the anchor pusher holder 196, and the needle holder 190, the anchor pusher holder 196 is able to move and propel towards, e.g. move closer to, the needle holder 190, and accordingly, the pair of anchors 144 (having the suture attached thereto), may be disengaged from the purse-string suture needles 140.

Referring to FIGS. 25E and 25F, the anchor pusher holder 196 may further include an anchor pusher hooking element 195, for example, a protrusion, formed on a part of the anchor pusher holder 196. The needle holder 190 may further include a needle holder hooking element 192, for example, a protrusion, formed on a part of the needle holder 190. In various embodiments, the needle holder hooking element 192 and the anchor pusher hooking element 195 may each be shaped and dimensioned to interact with each other to facilitate the retraction of the purse-string suture needles 140 from the distal end of the surgical device 100.

As shown in FIG. 25E, in the coupled position 150A, the anchor pusher hooking element 195 may be positioned adjacent to the needle holder hooking element 192. In the decoupled position 150B, the propulsion of the anchor pusher holder 196 to the needle holder 190 causes the anchor pusher hooking element 195 to be positioned away from the needle holder hooking element 192. Therefore, upon retraction of the purse-string suture needles 140, e.g. towards the proximal end 101a of the shaft 101, the anchor pusher hooking element 195 may “hook” onto the needle holder hooking element 192, to pull and retract the needle holder 190 carrying the pair of purse-string suture needles 140.

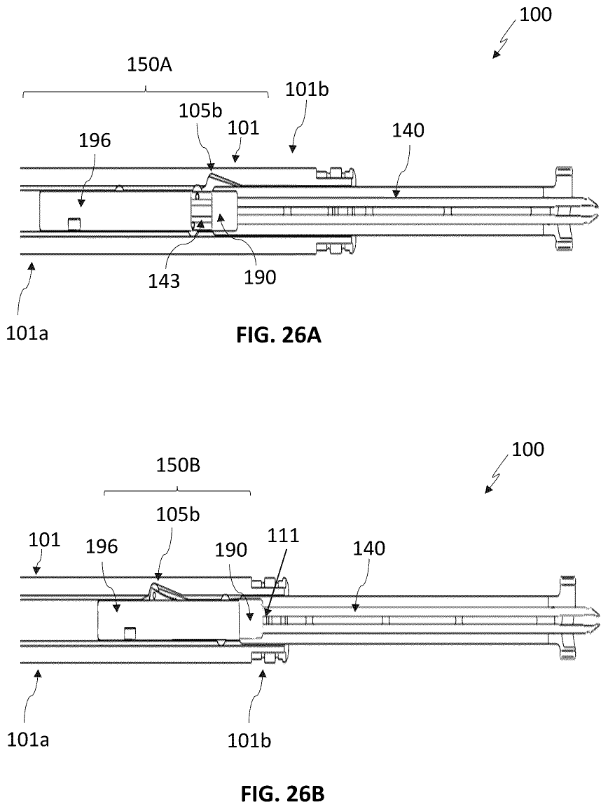

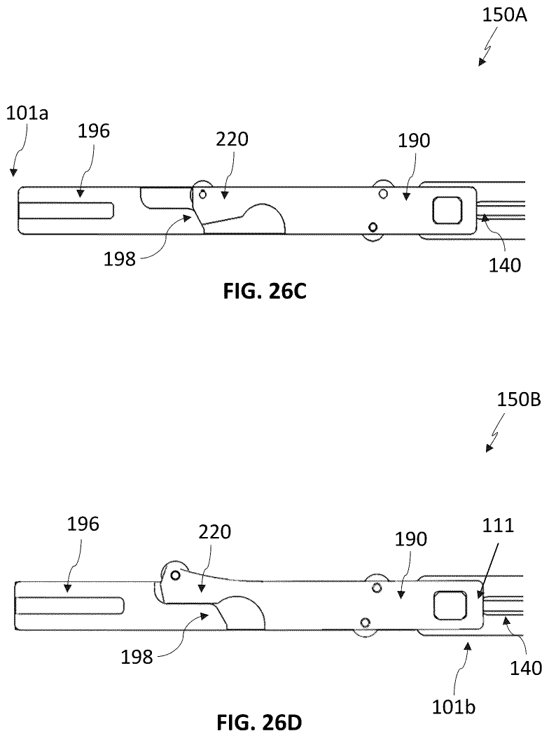

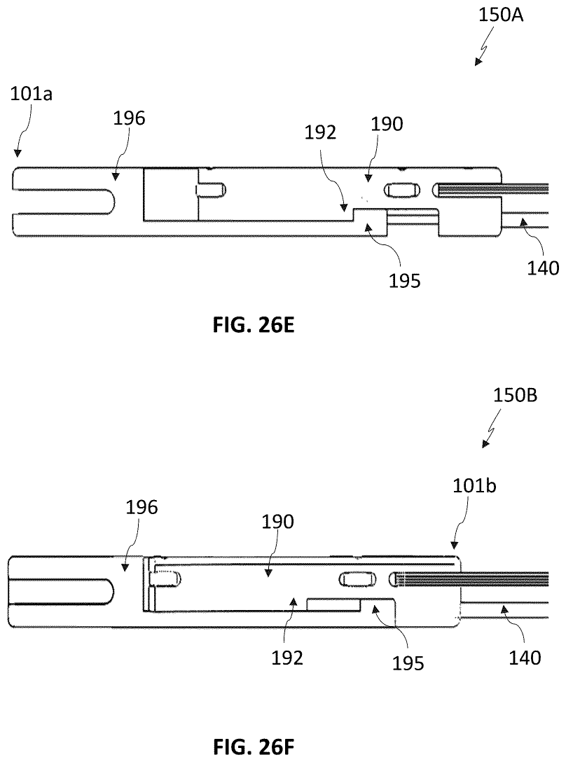

FIGS. 26A and 26B show side sectional views of the surgical device 100 (the clamp 102 is not shown) with particular emphasis on the decoupler assembly 150, in the coupled position 150A (FIG. 26A), and the decoupled position 150B (FIG. 26B), in accordance with another embodiment of the disclosure. FIGS. 26C and 26D show side views of the decoupler assembly 150 in the coupled position 150A (FIG. 26C) and the decoupled position 150B (FIG. 26D), from a side of the surgical device. FIGS. 26E and 26F show side views of the decoupler assembly 150 in the coupled position 150A (FIG. 26E) and the decoupled position 150B (FIG. 26F), from another side of the surgical device 100. FIGS. 26C and 26E corresponds to the views of the decoupler assembly 150 as shown in FIG. 26A, and FIGS. 26D and 26F corresponds to the view of the decoupler assembly 150 as shown in FIG. 26B, respectively. The decoupler assembly 150 as shown in FIGS. 26A to 26F may be based on the decoupler assembly 150 discussed with reference to FIGS. 25A to 25F, and repeated descriptions will be omitted.

Referring to FIGS. 26A to 26D, the needle holder 190 of the decoupler assembly 150 may include a first portion 220 which is pliable. For example, the first portion 220 may be flexible, clastic and bendable upon the application of a load. The first portion 220 may be positioned directly adjacent to the anchor pusher holder 196 in the coupled position 150A. The first portion 220 may be shaped and dimensioned to interact with the corresponding wedge-shaped portion 198 of the anchor pusher holder 196, and the inner shaft 152 of the shaft 101 may include a second recess 105b, e.g. void or cut-out formed in the inner shaft 152 of the shaft 101. The second recess 105b may be shaped and dimensioned to receive at least, the first portion 220 of the needle holder 190. In various embodiments, the restriction of the tunnel of the shaft 101 may prevent the first portion 220 from deforming, e.g. bending, as it moves towards the predetermined position 111. In some embodiments, a rolling element 109 may be formed on the needle holder 190 and/or the anchor pusher holder 196 to reduce friction as the needle holder 190 and/or the anchor pusher holder 196 moves along the longitudinal axis of the shaft 101.

Since the first portion 220 is pliable, a user may easily load the pair of anchor pushers 143, and the corresponding pair of anchors 144 (with the suture pre-attached thereto), in the hollow interior of the purse-string suture needles 140.

As the decoupler assembly 150 moves toward the predetermined position 111, e.g. from the proximal end 101a to the distal end 101b of the inner shaft 152 of the shaft 101, the wedged-shaped portion 198 of the anchor pusher holder 196 may push and move the needle holder 190 to the predetermined position 111, at which the decoupler assembly 150 may move into the decoupled position 150B. Due to the second recess 105b and because the first portion 220 is pliable, the first portion 220 may fold and bend into the second recess 105b. Accordingly, the bending of the first portion 220 creates a space to allow the anchor pusher holder 196 to propel towards the needle holder 190, thereby causing the disengagement of the pair of anchors 144 (having the suture attached thereto) from the purse-string suture needles 140. Alternatively, in some embodiments, the anchor pusher holder 196 may include a portion which is pliable, and adapted to fold and bend into the second recess 105b at the predetermined position 111.

Referring to FIGS. 26E and 26F, the anchor pusher holder 196 may further include an anchor pusher hooking element 195, and the needle holder 190 may further include a needle holder hooking element 192. The needle holder hooking element 192 and the anchor pusher hooking element 195 may cooperate to facilitate the retraction of the needle holder 190, as explained above.

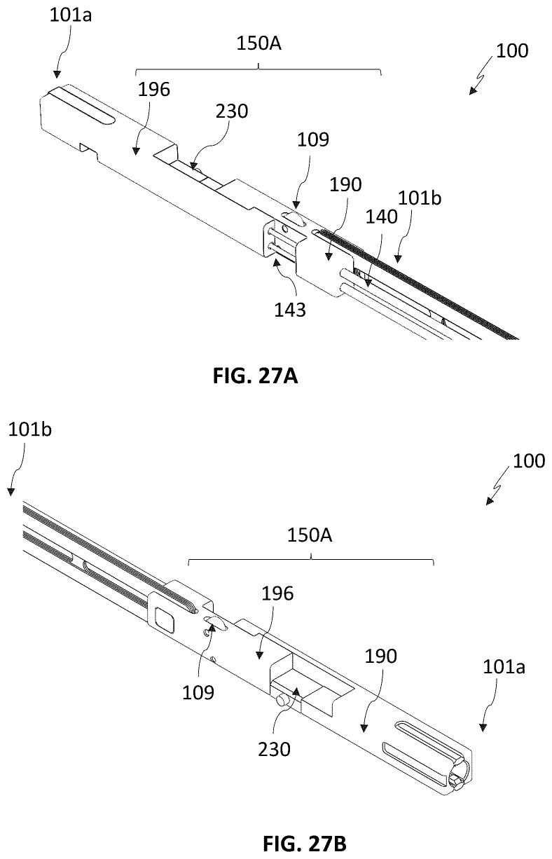

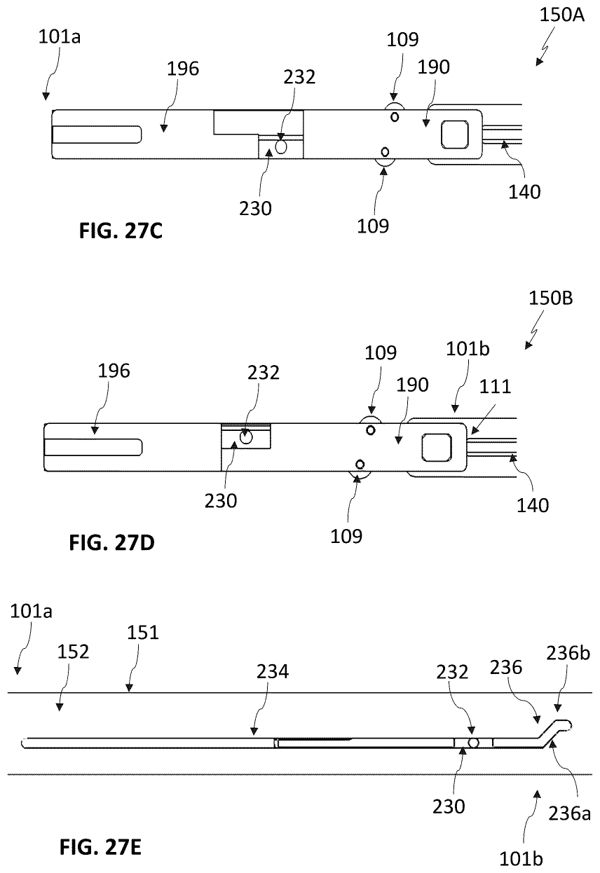

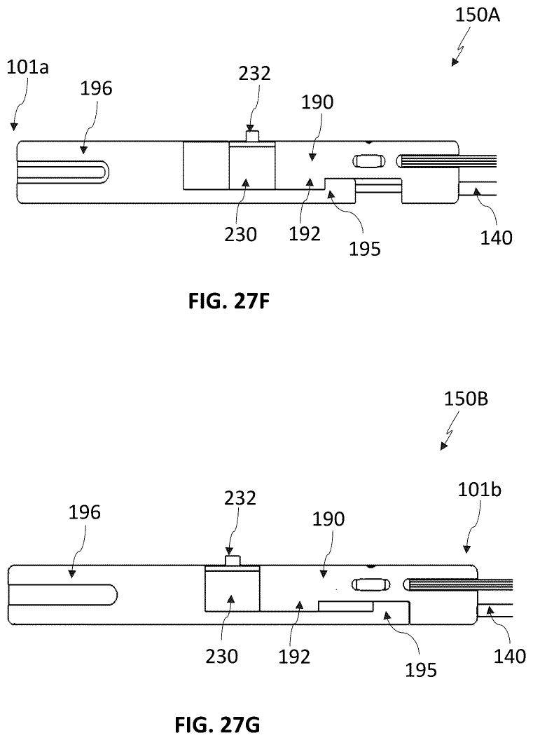

FIGS. 27A and 27B show perspective views of decoupler assembly 150 in the coupled position 150A, in accordance with another embodiment of the disclosure. FIGS. 27C and 27D show side views of the decoupler assembly 150 in the coupled position 150A (FIG. 27C) and the decoupled position 150B (FIG. 27D). FIG. 27E shows a side view of the decoupler assembly 150 the coupled position 150A, and the first guide rail 234, from a side of the surgical device 100. FIGS. 27F and 27G show side views of the decoupler assembly 150 in the coupled position 150A (FIG. 27F) and the decoupled position 150B (FIG. 27G), from another side of the surgical device 100. FIGS. 27C and 27F correspond to the views of the decoupler assembly 150 as shown in FIGS. 27A and 27B. FIG. 27G corresponds to the view of the decoupler assembly 150 as shown in FIG. 27D.

Referring to FIGS. 27A to 27E, the decoupler assembly 150 may include a second spacer 230 positioned immediately adjacent to the needle holder 190 and the anchor pusher holder 196. In other words, the needle holder 190 and the anchor pusher holder 196 may be spaced from each other via the second spacer 230 positioned therebetween. This facilitates the loading of the pair of anchor pushers 143, and the corresponding pair of anchors 144 (with the suture pre-attached thereto), in the hollow interior of the purse-string suture needles 140. The second spacer 230 may further include a first lug 232 arranged on a surface of the second spacer 230. The first lug 232 may be a protrusion extending from the second spacer 230.

Referring to FIG. 27E, the inner shaft 152 of the shaft 101 may include a first guide rail 234 extending along an inner surface and longitudinal axis of the inner shaft 152 of shaft 101. The first guide rail 234 may receive and permit the movement of the first lug 232 within the first guide rail 234. The first guide rail 234 may include a first cam 236 positioned along the length of the first guide rail 234. The first cam 236 may include a first bent portion 236a moving towards an elevated surface of the inner shaft 152 of shaft 101, to “lift” the first lug 232 sliding therein, and the first cam 236 may terminate at the end of the first bent portion 236a. For example, once the first lug 232 is elevated to a certain height, the anchor pusher holder 196 may be “unlocked”. In some embodiments, as shown in FIG. 27E, the first cam 236 may further include a first straight portion 236b, at which the first cam 236 terminates.

Referring to FIGS. 27C to 27E, as the decoupler assembly 150 moves toward the predetermined position 111, for example, by moving from the proximal end 101a to the distal end 101b of the shaft 101, the anchor pusher holder 196 may push and move the first lug 232 of the second spacer 230 along the first guide rail 234, and the first bent portion 236a of the first cam 236. At the predetermined position 111, the first lug 232 moves to the end of the first cam 236, thereby moving the decoupler assembly 150 into the decoupled position 150B. The movement of the first lug 232 of the second spacer 230 into the end of the first cam 236 creates a space to allow the anchor pusher holder 196 to propel towards the needle holder 190, thereby causing the disengagement of the pair of anchors 144 (having the suture attached thereto) from the purse-string suture needles 140.

Referring to FIGS. 27F and 27G, the anchor pusher holder 196 may further include an anchor pusher hooking element 195, and the needle holder 190 may further include a needle holder hooking element 192. The needle holder hooking element 192 and the anchor pusher hooking element 195 may cooperate to facilitate the retraction of the needle holder 190, as explained above.

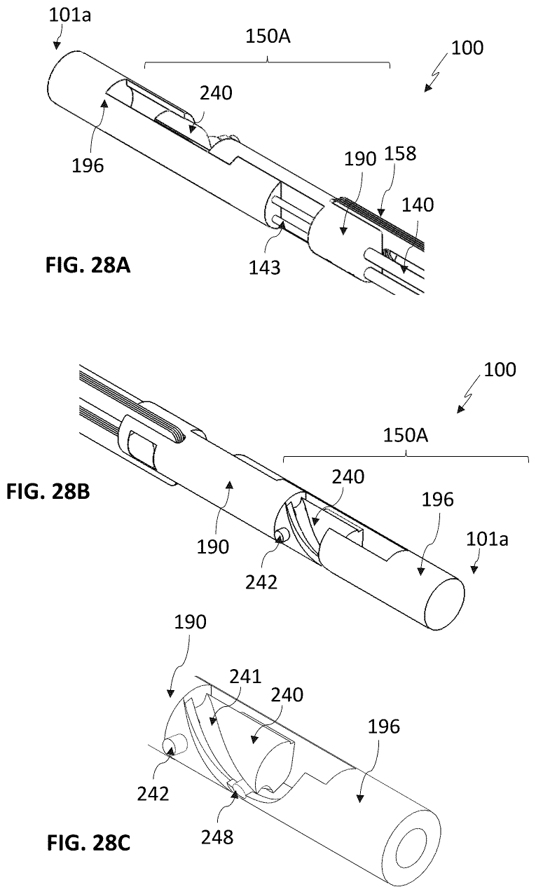

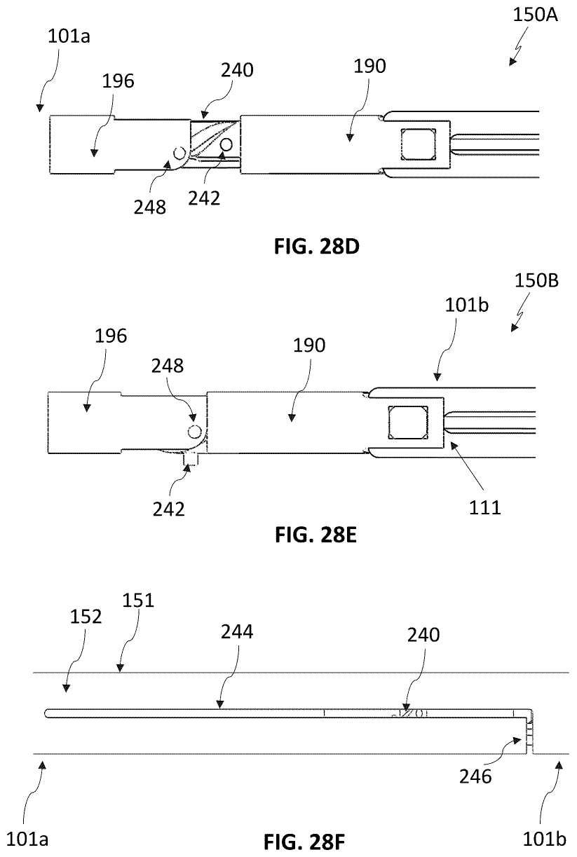

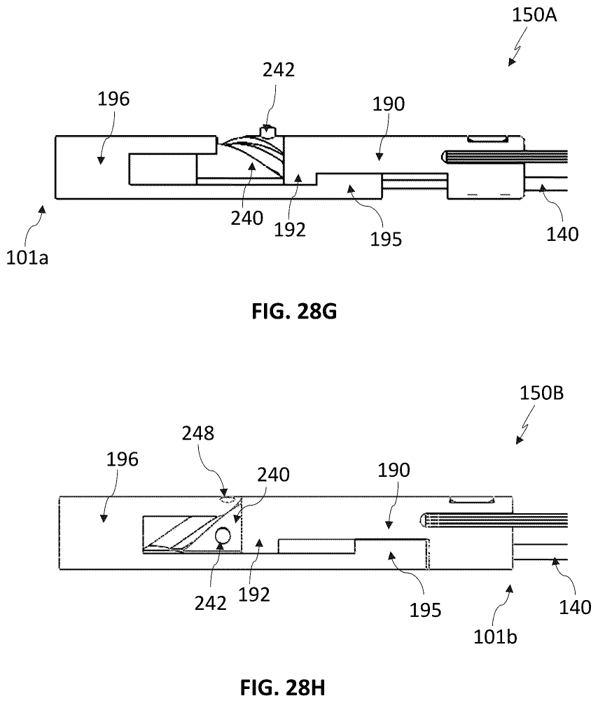

FIGS. 28A and 28B show perspective views of decoupler assembly 150 of the surgical device 100 in the coupled position 150A in accordance with another embodiment of the disclosure. FIG. 28C shows an enlarged sectional view of the third spacer 240 in the coupled position 150A. FIGS. 28D and 28E show side views of the decoupler assembly 150 in the coupled position 150A (FIG. 28D) and the decoupled position 150B (FIG. 28E). FIG. 28F shows a side view of the decoupler assembly 150 the decoupled position 150A and the second guide rail 244 from a side of the surgical device 100. FIGS. 28G and 28H show side views of the decoupler assembly 150 in the coupled position 150A (FIG. 28G) and the decoupled position 150B (FIG. 28H), from another side of the decoupler assembly of the surgical device 100. FIGS. 28D and 28G corresponds to the view of the decoupler assembly 150 as shown in FIGS. 28A and 28B. FIG. 28E corresponds to the view of the decoupler assembly 150 as shown in FIG. 28H.

Referring to FIGS. 28A to 28F, the decoupler assembly 150 may include a third spacer 240 positioned between the needle holder 190 and the anchor pusher holder 196, to space apart the needle holder 190 and the anchor pusher holder 196. This facilitates the loading of the pair of anchor pushers 143, and the corresponding pair of anchors 144 (with the suture pre-attached thereto), in the hollow interior 121 of the purse-string suture needles 140. The third spacer 240 can rotate along the third spacer 240's axis in the longitudinal direction, i.e. parallel to the longitudinal axis of the shaft 101. The third spacer 240 may include a curved surface 241, and further include a second lug 242 arranged on a surface of the third spacer 240. The second lug 242 may be a radial protrusion extending from the third spacer 240. The anchor pusher holder 196 may include a pushing lug 248 formed on a part of the anchor pusher holder 196. The pushing lug 248 may interact with the curved surface 241, and the second lug 242 may interact with the second guide rail 244 formed along the inner shaft 152 of the shaft 101, to facilitate the moving of the decoupler assembly 150 into the decoupled positioned 150B. It is contemplated that a matching curved surface with respect to the curved surface 241 of the third spacer 240, may be formed on the anchor pusher holder 196, instead of the aforementioned pushing lug 248.

Referring to FIG. 28F, the inner shaft 152 of the shaft 101 may include a second guide rail 244 extending along the longitudinal axis of the inner shaft 152 of the shaft 101. The second guide rail 244 may receive and permit the movement of the second lug 242 within the second guide rail 244, and may prevent the rotation of the third spacer 240 until the decoupler assembly 150 reaches the predetermined position 111 at the distal end 101b of the shaft 101. In various embodiments, the pushing lug 248 of the anchor pusher holder 196 may transmit an axial load or force to the second lug 242 of the third spacer 240. Since the second lug 242 may be contained in the second guide rail 244, the rotation of the third spacer 240 may be prevented until the decoupler assembly 150 reaches the predetermined position 111. The second guide rail 244 may include a rail recess 246 positioned along the length thereof, for example, at an end of the second guide rail 244. The second guide rail 244 may terminate at the rail recess 246.

Referring to FIGS. 28D to 28F, as the decoupler assembly 150 moves toward the predetermined position 111 at the distal end 101b of the shaft 101, the anchor pusher holder 196 may push and move the third spacer 240 via the interface formed between the pushing lug 248 and curved surface 241, e.g. the contact between the pushing lug 248 and curved surface 241, which in turn, moves the needle holder 190. Specifically, the second lug 242 may slide within the second guide rail 244. When the needle holder 190 moves to predetermined position 111, the rail recess 246 provides a space that permits the second lug 242 to move into the rail recess 246. The movement of the second lug 242 of the third spacer 240 into the rail recess 246 translates into the rotation of the third spacer 240 which allows the pushing lug 248 to move along the curved surface 241 of the third spacer 240, and thereby moving the decoupler assembly 150 into the decoupled position 150B. At the same time, e.g. in the same movement, the anchor pusher holder 196 propels towards the needle holder 190, thereby causing the disengagement of the pair of anchors 144 (having the suture attached thereto) from the purse-string suture needles 140. For example, the rotation of third spacer 240 allow linear movement of the anchor pusher holder 196 toward the needle holder 190.

Referring to FIGS. 28G and 28H, the anchor pusher holder 196 may further include an anchor pusher hooking element 195, and the needle holder 190 may further include a needle holder hooking element 192. The needle holder hooking element 192 and the anchor pusher hooking element 195 may cooperate to facilitate the retraction of the needle holder 190, as explained above.

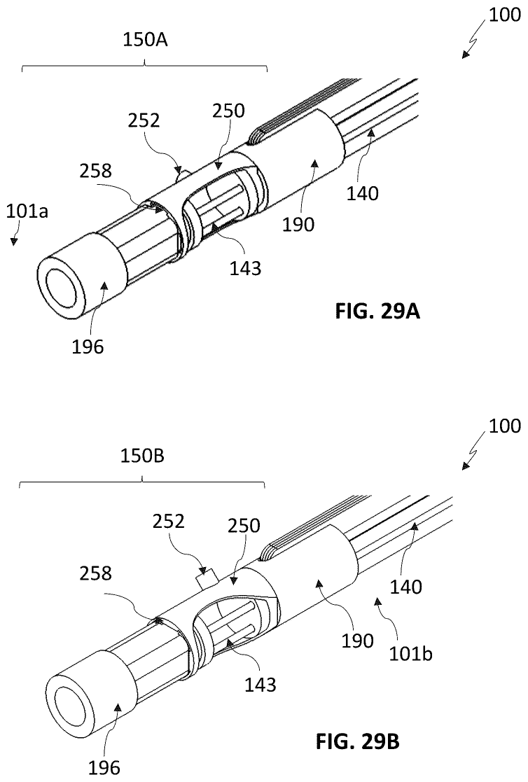

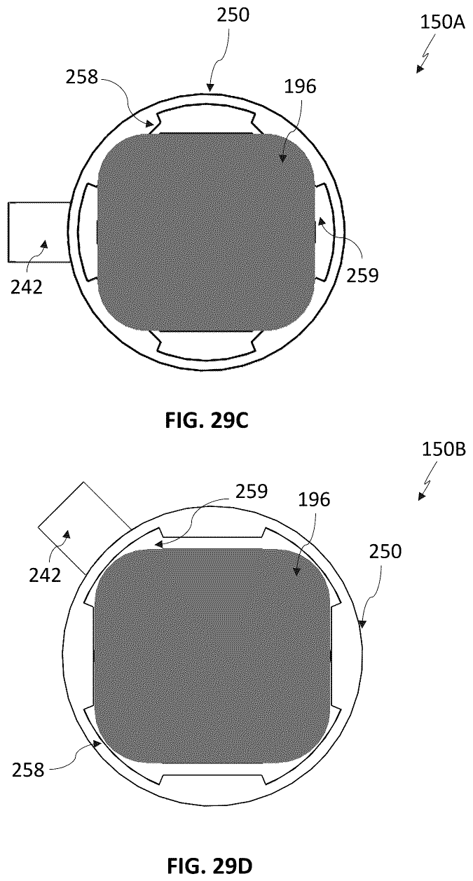

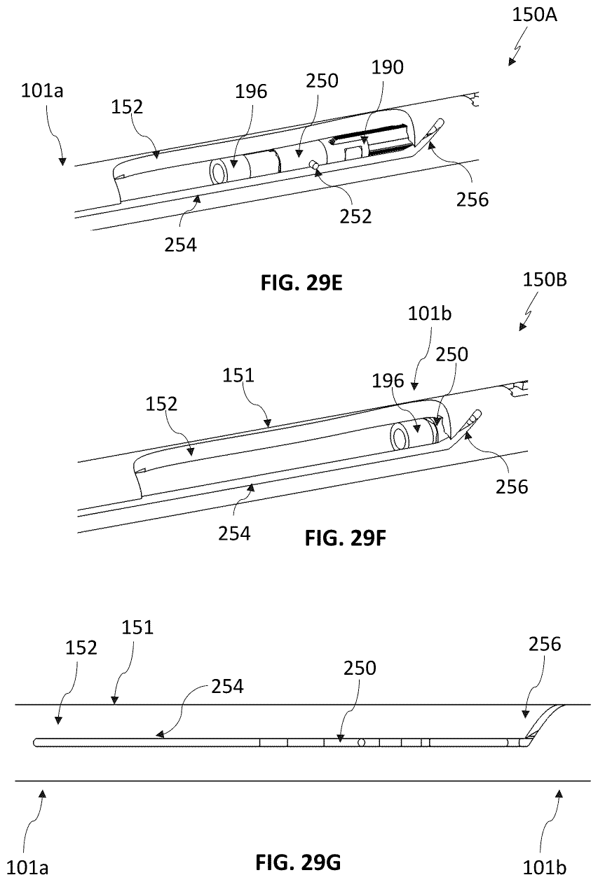

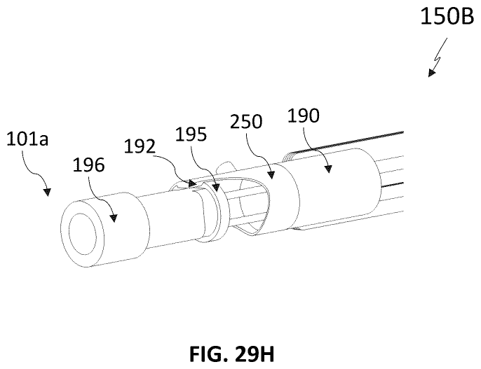

FIGS. 29A and 29B show perspective views of the decoupler assembly 150 of the surgical device 100 in the coupled position 150A (FIG. 29A), and the decoupled position 150B (FIG. 29B), in accordance with another embodiment of the disclosure. FIGS. 29C and 29D show sectional views of the engagement of the spacer lock 258 and the anchor pusher holder 196 in the coupled position 150A (FIG. 29C), and the decoupled position 150B (FIG. 29D). FIGS. 29E and 29F show perspective side sectional views of the decoupler assembly 150 in the coupled position 150A (FIG. 29E) and the decoupled position 150B (FIG. 29F). FIG. 29G shows a side view of the third guide rail 254, from a side of the surgical device 100. FIG. 29H shows a sectional view of the decoupler assembly 150 in the decoupled position 150B. FIGS. 29C and 29E correspond to the views of the decoupler assembly 150 as shown in FIG. 29A, and FIGS. 29D, 29F and 29H correspond to the view of the decoupler assembly 150 as shown in FIG. 29B.

Referring to FIGS. 29A to 29G, the decoupler assembly 150 may include a fourth spacer 250 positioned between the needle holder 190 and the anchor pusher holder 196, to space apart the needle holder 190 and the anchor pusher holder 196. The fourth spacer 250 may include a spacer lock 258 arranged on an outer surface of the fourth spacer 250, and may further include a third lug 252 arranged on the outer surface of the fourth spacer 250. The third lug 252 may be a radial protrusion extending from the fourth spacer 250. In the coupled position 150A as shown in FIG. 29C, the spacer lock 258 may be adapted to engage with a part of the anchor pusher holder 196. This creates a restricted passage 259 in which the anchor pusher holder 196 is unable to move toward the needle holder 190 and therefore “locks” the anchor pusher holder 196 as it slides along the longitudinal axis of the shaft 101. The spacer lock 258 may also cause the anchor pusher holder 196 to be positioned away from the needle holder 190, to facilitate the loading of the pair of anchor pushers 143, and the corresponding pair of anchors 144 (with the suture pre-attached thereto), in the hollow interior 121 of the purse-string suture needles 140. In addition, the anchor pusher holder 196 engages the spacer lock 258 so as to push the needle holder 190 through the fourth spacer 250 to the predetermined position 111 at the distal end 101b of the shaft 101.

As shown in FIGS. 29E to 29G, the inner shaft 152 of the shaft 101 may include a third guide rail 254 extending along an inner surface and longitudinal axis of the shaft 101. The third guide rail 254 may receive and permit the movement of the third lug 252 within the third guide rail 254, and may prevent the rotation of the fourth spacer 250 until the decoupler assembly 150 reaches the predetermined position 111. For example, the third guide rail 254 may facilitate the movement of the third lug 252 as the decoupler assembly 150 moves from the coupled position 150A to the decoupled position 150B, in which the third lug 252 moves within the third guide rail 254 from the proximal end 101a to the distal end 101b of the shaft 101. The third guide rail 254 may include a second cam 256 positioned along the length thereof, for example, at an end of the third guide rail 254. The second cam 256 may be adapted to extend at an angle, relative to the third guide rail 254, and the third guide rail 254 may terminate at the second cam 256.

Referring to FIGS. 29C to 29G, as the decoupler assembly 150 moves toward the predetermined position 111, the anchor pusher holder 196 may engage with the spacer lock 258, and may push and move the fourth spacer 250, and the needle holder 190 along the longitudinal axis of the shaft 101. Specifically, the third lug 252 may slide within the third guide rail 254 to prevent the rotation of the fourth spacer 250 until the decoupler assembly 150 reaches the predetermined position 111. At the predetermined position 111, the third lug 252 may slide into the second cam 256, thereby moving the decoupler assembly 150 into the decoupled position 150B. The sliding of the third lug 252 of the fourth spacer 250 into the second cam 256 results in the rotation of the fourth spacer 250, and the disengagement of the spacer lock 258 and the part of the anchor pusher holder 196. As shown in FIG. 29D, this removes the restriction and creates a passage 259 which allows the anchor pusher holder 196 to freely propel towards the needle holder 190, thereby causing the disengagement of the pair of anchors 144 (having the suture attached thereto) from the purse-string suture needles 140. For example, the rotation of the third lug 252 creates a free passage 259 to allow the anchor pusher holder 196 to move toward the needle holder 190.

Referring to FIGS. 29H, the anchor pusher holder 196 may further include an anchor pusher hooking element 195, and the needle holder 190 may further include a needle holder hooking element 192. The needle holder hooking element 192 and the anchor pusher hooking element 195 may cooperate to facilitate the retraction of the needle holder 190, as explained above.

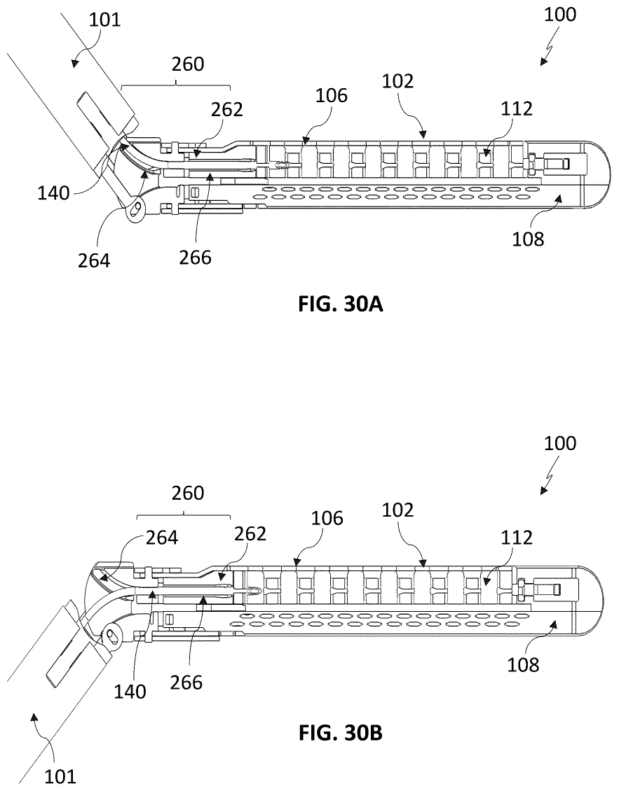

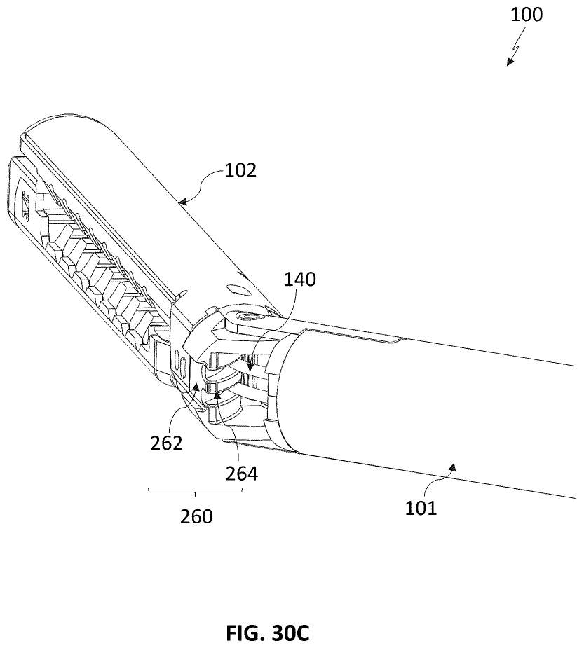

FIGS. 30A to 30C show sectional views of the surgical device 100, with particular emphasis on an articulation assembly 260 of the surgical device 100. The surgical device 100 may further include an articulation assembly 260 positioned at the coupling of the shaft 101 and the clamp 102 of the surgical device 100, for example, proximal or at the articulation joint at which the shaft 101 is coupled to the clamp 102. In various embodiments, the articulation assembly 260 may include an articulation block 262. The articulation block 262 may be positioned on either the first jaw 103 and/or the second jaw 104. In one embodiment, the articulation block 262 may be formed on the first 106 longitudinal portion of the second jaw 104 of the clamp 102, and the articulation block 262 may be positioned adjacent to the purse-string suture profile 112 of the clamp 102.

As shown in FIGS. 30A to 30C, the articulation assembly 260 may include articulation recesses 264, e.g. a pair of articulation recesses 264 which is connected to and leads into linear passages, e.g. a pair of linear passages 266, the articulation recesses 264 and linear passages 266 adapted to receive the purse-string suture needles 140. In other words, the articulation recesses 264 may be connected to the linear portions 266 to provide a path for the purse-string suture needles 140 to pass from the shaft 101 to the distal end of the surgical device 100.

In various embodiments, the clamp 102 may be rotatable with respect to the shaft 101, and may therefore be positioned at an angle with respect to the shaft 101. This forms a bend at the articulation joint of the surgical device 100. As the purse-string suture needles 140 are urged through the surgical device 100, the purse-string suture needles 140 which comprise a super-elastic material, may bend at the articulation joint. The articulation recesses 264 may provide a space to accommodate the bending of the purse-string suture needles 140 when the clamp 102 is positioned at an angle, e.g. rotated, with respect to the shaft 101. The articulation recesses 264 lead into the linear passages 266 which provides a linear path adapted to guide the pair of purse-string needles 140 to move in a linear manner as they advance towards and through the purse-string suture profile 112 of the first longitudinal portion 106.

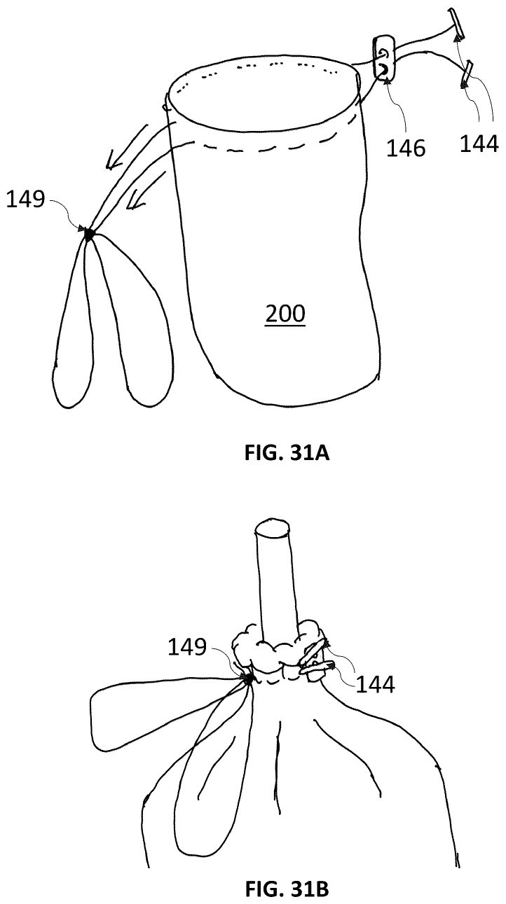

FIGS. 31A and 31B illustrate a purse-string suture formed around a tubular tissue 200, for example a mammalian colon. A suture fastener 149 may be used to work with the purse-string suture. The suture fastener 149 allows both the anchors 144 connected to the suture to retract independently. The suture fastener 149 can tighten sufficiently to provide adequate frictional force on the suture such that the tightened purse-string suture maintains a tightened state.

The suture fastener 149 can be added to facilitate surgery and minimize surgery time. In some embodiments, the suture fastener 149 may include a pre-made knot on using suture, or a separate component to detent suture movement through it.

In some embodiments, the suture fastener(s) 149 may be formed of or from a rigid material. Alternatively, the suture fastener(s) 149 may be formed of or from a non-rigid material, or an clastic material.

In some embodiments, the suture fastener 149 can be a separate component. The separate components form one or two orifice(s) through which the suture may slidably pass the orifice(s). The orifice(s) may exert a frictional force on the suture, such that it will keep the suture in place when a tensional force is applied through the suture by the tightened purse-string suture. The fastener may be able to allow the suture to slide from both end of the loop. In some embodiments, the fastener can also be in the form of a knot made by the suture. The knot may be formed by routing the suture in a certain way such that it can exert friction force on the looping suture from both ends.

In some embodiments, there comprises an anchor stopper quick release mechanism operable to hold the anchor stopper 146 securely to the first jaw 103 or second jaw 104. In some embodiments, the anchor stopper 146 may be resilient to mechanical vibration and mechanical shock. The quick-release mechanism allows the anchor stopper 146 to be released only after the purse-string suture needles 140 puncture through the anchor stopper 146 and stays in releasing state thereafter. This allows the anchor stopper 146 to be released from the jaw easier.

While the surgical device 100 described above may be particularly suited for performing tissue resection, suturing and stapling, it is contemplated that surgical device 100 may be implemented to only include the first longitudinal portion 106 comprising the purse-string suture profile 112, and the blade assembly. In this embodiment, the surgical device 100 may be adapted to perform tissue resection and suturing. It is further contemplated that the surgical device 100 may be implemented to only include the first longitudinal portion 106 comprising the purse-string suture profile 112, and the surgical device 100 may be adapted to only perform tissue suturing.

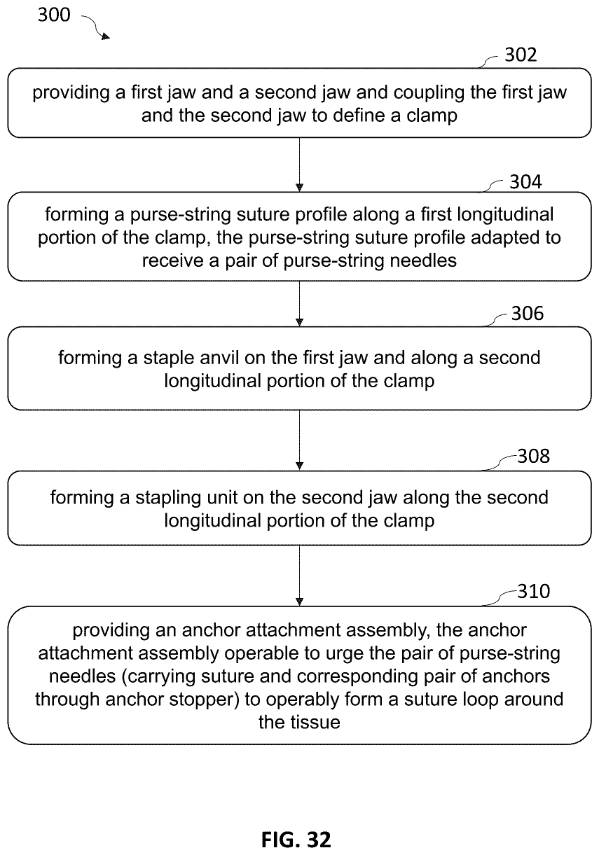

According to another aspect of the disclosure and with reference to FIG. 32, there is a method 300 of manufacturing a surgical device 100. The surgical device 100 may refer to the surgical device 100 described with reference to FIGS. 1 to 31B and repeated description will be omitted for brevity. The method 300 comprises the steps of
Step 302: providing a first jaw and a second jaw and coupling the first jaw and the second jaw to define a clamp;
Step 304: forming a purse-string suture profile along a first longitudinal portion of the clamp, the purse-string suture profile adapted to receive a pair of purse-string suture needles;
Step 306: forming a staple anvil on the first jaw and along a second longitudinal portion of the clamp;
Step 308: forming a stapling unit on the second jaw along the second longitudinal portion of the clamp; and
Step 310: providing an anchor attachment assembly, the anchor attachment assembly operable to urge the pair of purse-string needles (carrying suture and corresponding pair of anchors through anchor stopper) to operably form a suture loop around the tissue.

In some embodiments, the formation of the anchorage includes forming a suture loop around the tissue.

In various embodiments, method 300 further comprises, providing a shaft, the shaft having a hollow interior for housing the pair of suture needles, and pivotably coupling the shaft to the clamp in a manner such the clamp is rotatable with respect to the shaft.

In various embodiments, method 300 further comprises, configuring the shaft to hold a first part of the anchor attachment assembly. The first part of the anchor attachment assembly may comprise a pair of anchor pushers adapted to constrain the corresponding pair of anchors, and to disengage the pair of purse-string suture needles from the corresponding pair of anchors.

In various embodiments, the first part of the anchor attachment assembly may further comprise a decoupler assembly, comprising a needle holder coupled to the pair of purse-string suture needles; an anchor pusher holder positioned proximal to the needle holder, the anchor pusher holder coupled to the pair of anchor pushers, wherein the decoupler assembly is operable to move (e.g. via a sliding movement) to a predetermined position along a longitudinal axis of the shaft, at which the anchor pusher holder propels towards the needle holder, thereby disengaging the corresponding pair of anchors from the pair of purse-string suture needles. Various embodiments of the decoupler assembly have been discussed above and repeated descriptions are omitted for brevity.

In various embodiments, method 300 further comprises, providing a second part of the anchor attachment assembly, and positioning the second part of the anchor attachment assembly on the second jaw and adjacent to the purse-string suture profile. The second part of the anchor attachment assembly may comprise an anchor stopper operable to be pierced by the pair of purse-string needles, the anchor stopper configured to interact with the corresponding pair of anchors and the suture to form a purse-string suture loop. Various embodiments of the anchor stopper have been discussed above and repeated descriptions are omitted for brevity.

In various embodiments, the second part of the anchor attachment assembly may further comprise an anchor chamber positioned on the second jaw along the first longitudinal portion of the clamp and adjacent to the purse-string suture profile, the anchor chamber for receiving and accommodating the corresponding pair of anchors. Various embodiments of the anchors have been discussed above and repeated descriptions are omitted for brevity.

In various embodiments, the pair of purse-string suture needles is formed from or of a nickel-titanium alloy. Various embodiments of the purse-string suture needles have been discussed above and repeated descriptions are omitted for brevity.

In various embodiments, the purse-string suture profile formed along the first longitudinal profile of the clamp comprises an upper suture profile formed along the first jaw, and a lower suture profile formed along the second jaw, wherein the upper purse-string suture profile and lower purse-string suture profile each comprise a plurality of ridges and a plurality of troughs, each trough of the plurality of troughs positioned between respective ridges of the plurality of ridges. Various embodiments of the purse-string suture profile have been discussed above and repeated descriptions are omitted for brevity.

In various embodiments, method 300 further comprises, providing a blade assembly, the blade assembly comprising an actuator and a blade, the blade slidably configured to move the clamp between an open position and a clamped position, and simultaneously extend along the longitudinal axis of the clamp, the blade operable to incise the tissue when the clamp is in the clamped position. In some embodiments, the blade is arranged between the first longitudinal portion and the second longitudinal portion.

In various embodiments, method 300 further comprises, providing a suture fastener configured to facilitate closure of the purse-string suture loop from at least one end of a suture loop.

In various embodiments, method 300 further comprises, providing an articulation assembly configured to be positioned at the coupling of the shaft and the clamp, the articulation assembly comprising an articulation block positioned on the second jaw, adjacent to the purse-string suture profile of the clamp, wherein the articulation block comprises a pair of articulation recesses leading into a pair of linear passages, the pair of articulation recesses and the pair of linear passages adapted to receive the pair of purse-string suture needles and guide the pair of purse-string suture needles to move in a linear manner towards the purse-string suture profile.

In various embodiments, method 300 further comprises, providing a connector interface for removably attaching the surgical device to a robotic instrument or a handle. Alternatively, in various other embodiments, the surgical device may be manufactured as a standalone device. For example, the surgical device may be integrated to a hand-held device or to a robotic instrument, without the requirement of a connector interface.

It is appreciable that the various steps 304, 306, 308, 310, and any of the above additional steps of method 300, may not necessarily be in the sequence as depicted, and may in some embodiments, be performed simultaneously. In other words, the order of the steps 304, 306, 308, 310 and the additional steps of method 300 as defined above, may be changed. In some embodiments, said steps may be performed at the same time.

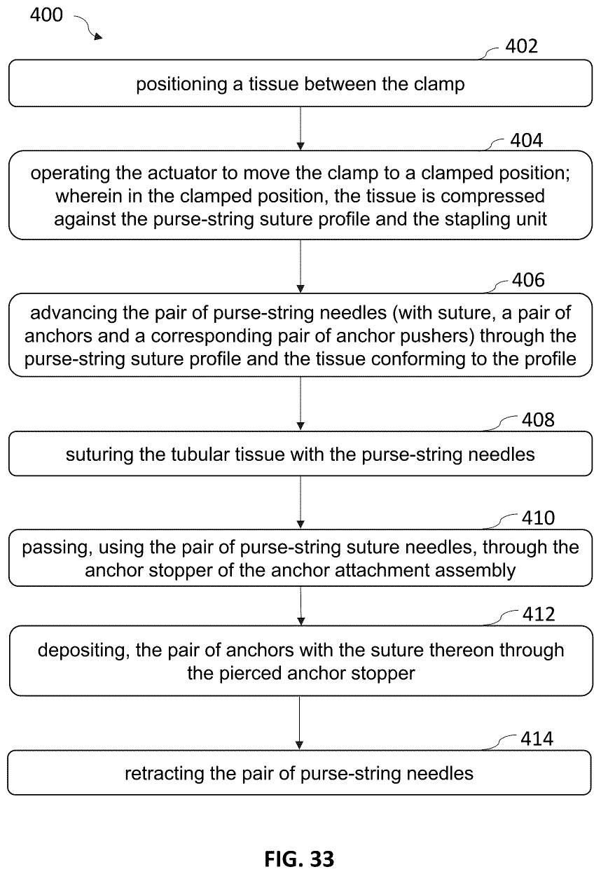

According to another aspect of the disclosure and with reference to FIG. 33, there is a method 400 of using the surgical device 100 to perform suturing of a tubular tissue, comprising
Step 402: positioning a tissue between the clamp;
Step 404: operating the blade to move the clamp to a clamped position; wherein in the clamped position, the tissue is compressed against the purse-string suture profile and the stapling unit;
Step 406: advancing the pair of purse-string suture needles (with suture, a pair of anchors and corresponding anchor pushers) through the purse-string suture profile and through the tissue conforming to the profile;
Step 408: suturing the tubular tissue with the purse-string suture needles;
Step 410: passing, using the pair of purse-string suture needles, through the anchor stopper of the anchor attachment assembly;
Step 412: depositing, the pair of anchors with the suture thereon through the pierced anchor stopper, or through a corresponding pair of pre-made holes on the anchor stopper; and
Step 414: retracting the pair of purse-string suture needles.

In some embodiments, the purse-string suture needles may be hollow and contain the suture, the pair of anchors (which may have the suture pre-attached), and the anchor pushers therein. In step 406, the pair of purse-string suture needles, with suture, a pair of anchors and corresponding anchor pushers contained therein, may then be simultaneously passed through the purse-string suture profile and the tissue conforming to the profile.

In some embodiments, step 404 consists of closing the clamp 102 by sliding the blade 158 of the blade assembly, distally from the hinge 107. The blade 158 may be configured such that moving the blade 158 will urge the first and second jaws to close them. In step 404, as the clamp 102 is closed, the tissue is compressed and conforms to the purse-string suture profile 112 and the stapling unit 134.

In some embodiments of step 406, as the articulatable blade 158 is advancing towards the distal end of the clamp 102, the pair of purse-string suture needles 140 will advance together with the blade 158. As the tissue conforms to the purse-string suture profile 112, the purse-string suture needles 140 are able to pierce or penetrate the plied tissue sequentially as part of the suturing process.

In step 406, as the articulable blade 158 is advancing towards the distal end of the clamp 102, a staple ramp may be pushed forward in the same action, the staple ramp will push the staple pusher and in turn release one or more staples out of the stapling unit cartridge and the released staples will be plied against the staple anvil 132, whereby the staples are deformed such that they will penetrate the tissue and staple two or more layers of tissue together.

As the articulatable blade 158 advances towards the distal end of the surgical device 100, the edge of the blade 158 will operate to cut the tissue.

In step 408, when the purse-string suture needles advance and penetrate the anchor stoppers 146, the anchors 144 may be plied to advance further to detach from the purse-string suture needles. In some embodiments, the detachment may include sliding out of the interior of purse-string suture needles 140.

In step 410, passing the pair of purse-string suture needles 140 through the anchor stopper 146 may include piercing the pair of purse-string suture needles 140 through the pre-made holes 182, the Y-shaped slits 184 and/or the first section 186 formed on the anchor stopper body 180.

In step 412, after the articulatable blade 158 and purse-string suture needles 140 reaches the designed position, they may be retracted entirely, leaving the anchors 144 on the distal end of the anchor stoppers 146 and the suture in the tissue.

The clamp 102 can be opened and the resected tissue with suture formed in the tissue, the anchor stopper 146 and anchors 144 can be released from between the first jaw 103 and the second jaw 104. The stapling, cutting, and purse-string suture forming is completed.

It is appreciable that some steps of method 400 may be combined. It is further appreciable some steps of method 400 may take place in sequence or simultaneously. For example, steps 404, 406, 408 and 410 may take place simultaneously.

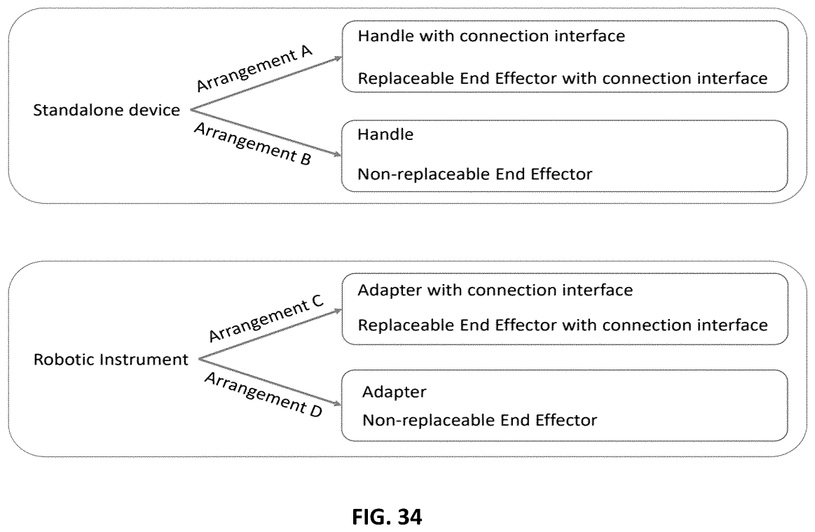

In some embodiments as shown in FIG. 34, the surgical device 100 may be interfaced with a handle to form a standalone device or part of a surgical system. The surgical device 100 may be detachable from the handle or may be integrally molded or formed with the handle. In some embodiments, the handle may be shaped and dimensioned for a medical practitioner, for example a surgeon, to manipulate the device by hand.

In some embodiments, the handle may include an interface to receive the connection interface from a replaceable surgical device, such that the replaceable surgical device can be detachably connected to the handle, and establish mechanical links with the mechanism in the handle.

In some embodiments, a robotic surgical system instrument may be interfaced with the surgical device 100. The robotic surgical system instrument may consist of a robotic system adapter and a replaceable or non-replaceable surgical device 100.

In some embodiments, the surgical device 100 may comprise an interface adapter to connect to the robotic surgical system instrument. Through the interface adapter, the surgical device 100 can establish mechanical links to secure the instrument on the robotic system and establish mechanical links to manipulate the surgical device 100.

In some embodiments, the adapter may comprise a second interface to connect to the surgical device 100 in the form of a replaceable end effector. The second interface can establish mechanical links to secure the replaceable end effector and establish mechanical links to manipulate the end effector.

In some embodiments, the adapter has an interface to connect to a robotic system. Through the interface, the adapter can establish mechanical links to secure the surgical device 100 on the robotic system and establish mechanical links to manipulate the surgical device in the form of an end effector. In other words, the adapter may be integrated with the non-replaceable end effector.

In some embodiments, the surgical device 100 may comprise a standalone device, and the surgical device 100 may be integrated to a hand-held device or to a robotic instrument, without the requirement of a connector interface.

According to another aspect of the disclosure, there is provided a kit of parts of a surgical device assembly including a surgical device, a pair of purse-string suture needles, at least one staple cartridge, and a connector interface. In various embodiments, the pair of purse-string suture needles may be formed of or from nickel-titanium alloy (nitinol). It is contemplated the purse-string suture needles may be formed from or of other super-elastic material.

In another aspect or embodiment, the surgical device may be implemented in the form of an end effector, the end effector comprising an interface that may be configured as a plug-and-use device with a handle or a robotic arm. Alternatively, in another aspect or embodiment, the surgical device may be a standalone device, and integrally formed with a handle or robotic instrument.

While the disclosure has been particularly shown and described with reference to specific embodiments, it should be understood by those skilled in the art that various changes in form and detail may be made therein without departing from the spirit and scope of the disclosure as defined by the appended claims of invention. The scope of the disclosure is thus indicated by the appended claims and all changes which come within the meaning and range of equivalency of the claims are therefore intended to be embraced.

1. A surgical device, the surgical device comprises
a first jaw coupled with a second jaw to define a clamp for receiving a tissue;
a purse-string suture profile formed along a first longitudinal portion of the clamp, the purse-string suture profile adapted to receive a pair of purse-string suture needles;
a staple anvil formed on the first jaw and along a second longitudinal portion of the clamp;
a stapling unit formed on the second jaw along the second longitudinal portion of the clamp; and
an anchor attachment assembly operable to urge the pair of purse-string suture needles to deliver a suture through the tissue to form an anchorage,
wherein a portion of the anchor attachment assembly is positioned on the second jaw and adjacent to the purse-string suture profile, and
wherein a portion of the anchor attachment assembly comprises an anchor stopper operable to be pierced by the pair of purse-string suture needles, the anchor stopper configured to interact with a corresponding pair of anchors and the suture to form a purse-string suture loop.

2. The surgical device of claim 1, further comprises a shaft, the shaft having a hollow interior for housing the pair of purse-string suture needles, the shaft pivotably coupled to the clamp in a manner such the clamp is rotatable with respect to the shaft.

3. The surgical device of claim 2, wherein the shaft is configured to hold a first part of the anchor attachment assembly.

4. The surgical device of claim 3, wherein the first part of the anchor attachment assembly comprises a pair of anchor pushers adapted to constrain the corresponding pair of anchors, and to disengage the pair of purse-string suture needles from the corresponding pair of anchors.

5. The surgical device of claim 4, wherein the pair of purse-string suture needles each comprise a hollow interior, and the corresponding pair of anchors are each pre-attached to the suture, and wherein the pair of anchor pushers, the corresponding pair of anchors, and the suture are contained within the hollow interior to deliver suture bonded anchors through the tissue to form the anchorage.

6. The surgical device of claim 4, wherein the first part of the anchor attachment assembly further comprises a decoupler assembly, comprising
a needle holder coupled to the pair of purse-string suture needles;
an anchor pusher holder positioned proximal to the needle holder, the anchor pusher holder coupled to the pair of anchor pushers,
wherein the decoupler assembly is operable to move to a predetermined position along a longitudinal axis of the shaft, at which the anchor pusher holder propels towards the needle holder, thereby disengaging the corresponding pair of anchors from the pair of purse-string suture needles.

7. The surgical device of claim 1, wherein the anchor stopper comprises a portion, shaped and dimensioned to receive the pair of purse-string suture needles.

8. The surgical device of claim 1, wherein the second part of the anchor attachment assembly comprises an anchor chamber positioned on the second jaw along the first longitudinal portion of the clamp and adjacent to the purse-string suture profile, the anchor chamber for receiving and accommodating the corresponding pair of anchors.

9. The surgical device of claim 4, wherein the corresponding pair of anchors each comprise an anchor body having a hollow interior operable to permit the suture to be threaded and bonded therethrough.

10. The surgical device of claim 1, wherein the pair of purse-string suture needles is formed from or of a nickel-titanium alloy (nitinol).

11. The surgical device of claim 1, wherein the purse-string suture profile formed along the first longitudinal portion of the clamp comprises an upper suture profile formed along the first jaw, and a lower suture profile formed along the second jaw,
wherein the upper suture profile and lower suture profile each comprise a plurality of ridges and a plurality of troughs, each trough of the plurality of troughs positioned between respective ridges of the plurality of ridges.

12. The surgical device of claim 2, wherein the shaft is pivotably coupled to the clamp via an articulation hinge.

13. The surgical device of claim 1, further comprises a blade assembly, the blade assembly comprises an actuator and a blade, the blade slidably configured to move the clamp between an open position and a clamped position, and simultaneously extend along the longitudinal axis of the clamp, the blade operable to incise the tissue when the clamp is in the clamped position.

14. The surgical device of claim 13, wherein the blade is arranged between the first longitudinal portion and the second longitudinal portion.

15. The surgical device of claim 1, further comprising
an articulation assembly positioned at the coupling of the shaft and the clamp, the articulation assembly comprising an articulation block positioned either on the first jaw and/or the second jaw, the articulation block positioned adjacent to the purse-string suture profile of the clamp,
wherein the articulation block comprises articulation recesses leading into linear passages, the articulation recesses and linear passages adapted to receive the purse-string suture needles and guide the purse-string suture needles to move towards and through the purse-string suture profile.

16. The surgical device of claim 1, further comprising a connector interface for removably attaching the surgical device to a robotic instrument or a handle.

17. A robotic instrument comprising a connector interface for receiving the surgical device of claim 1, wherein the surgical device is configured as an end effector.

18. A hand-held instrument comprising a connector interface for receiving the surgical device of claim 1, wherein the surgical device is configured as an end effector.

19. A method of manufacturing a surgical device, the method comprising the steps of
providing a first jaw and a second jaw and coupling the first jaw and the second jaw to define a clamp;
forming a purse-string suture profile along a first longitudinal portion of the clamp, the purse-string suture profile adapted to receive a pair of purse-string suture needles;
forming a staple anvil on the first jaw and along a second longitudinal portion of the clamp;
forming a stapling unit on the second jaw along the second longitudinal portion of the clamp; and
providing an anchor attachment assembly, the anchor attachment assembly operable to urge the pair of purse-string suture needles to deliver a suture through a tissue to form an anchorage,
wherein the method further comprises, providing a second part of the anchor attachment assembly and positioning the second part of the anchor attachment assembly on the second jaw and adjacent to the purse-string suture profile,
wherein the second part of the anchor attachment assembly comprises an anchor stopper operable to be pierced by the pair of purse-string needles, the anchor stopper configured to interact with a corresponding pair of anchors and the suture to form a purse-string suture loop.

20. A method of using the surgical device of claim 1 to perform suturing of a tubular tissue, comprising:
positioning a tissue between the clamp;
operating the blade to move the clamp to a clamped position; wherein in the clamped position, the tissue is compressed against the purse-string suture profile and the stapling unit;
advancing the pair of purse-string suture needles (with suture, the corresponding pair of anchors, and the pair of anchor pushers) through the purse-string suture profile;
suturing the tubular tissue with the purse-string suture needles;
passing, using the pair of purse-string suture needles, through the anchor stopper of the anchor attachment assembly;
depositing the corresponding pair of anchors with suture thereon through the pierced anchor stopper, and
retracting the pair of purse-string suture needles.
